SEQUENCE LISTING (15 sequences)

SEQ ID NO: 1 - DNA, 1569 bp, Mus musculus
  source 1..1569: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 2 - DNA, 1148 bp, Mus musculus
  source 1..1148: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgtcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 3 - DNA, 780 bp, Mus musculus
  source 1..780: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 4 - DNA, 388 bp, Mus musculus
  source 1..388: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 5 - protein, 307 aa, Homo sapiens
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQNETPQPIYQNSNVATKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 6 - protein, 280 aa, Homo sapiens
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQNETPQPIYQNSNVATKPTAAENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 7 - DNA, 924 bp, Mus musculus
  source 1..924: mol_type = other DNA
atgaccgagaccacaaagacccacgttatcctgctggcctgcggcagcttcaatcccatcactaaagggcacattcagatgttcgagagagccagggattatctgcacaagactggaagatttattgtgattggcgggattgtctctccggtccatgactcctacggaaaacagggccttgtgtcaagtcggcaccgtctcatcatgtgtcagctggctgtccagaattccgactggatcagggtggacccatgggagtgctatcaggacacctggcagacaacctgcagtgtgttggagcaccatcgagacctgatgaagagggtgaccggctgcatcctctccaacgtcaacacaccttccatgacacctgtgatcggacagccacagcatgagaacacccagcccatttaccagaacagcaatgtgcccaccaagcccactgcagccaagatcttgggaaaggtgggagaaagcctcagccggatctgctgcgtccgcccaccagtggagcgcttcacttttgtagatgagaacgccaacctgggcacagtgatgcggtatgaggagatcgagctgcgcatcttgctgctgtgtggtagtgacctgctggagtctttctgcatcccaggactctggaatgaggcagatatggaagtgattgttggggactttgggatcgtcgtggtacccagggatgcagcggacacagaccggatcatgaatcactcctccatactccgcaagtacaaaaacaacatcatggttgtgaaggatgatatcaaccatcccatgtctgtagtcagctccaccaagagcaggctggccctgcagcatggggacggccatgttgtggattacctgtcccagccagtcatcgattacatcctcaagagtcagctgtacatcaacgcctcgggctag

SEQ ID NO: 8 - protein, 307 aa, Mus musculus
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQHENTQPIYQNSNVPTKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 9 - DNA, 843 bp, Mus musculus
  source 1..843: mol_type = other DNA
atgaccgagaccacaaagacccacgttatcctgctggcctgcggcagcttcaatcccatcactaaagggcacattcagatgttcgagagagccagggattatctgcacaagactggaagatttattgtgattggcgggattgtctctccggtccatgactcctacggaaaacagggccttgtgtcaagtcggcaccgtctcatcatgtgtcagctggctgtccagaattccgactggatcagggtggacccatgggagtgctatcaggacacctggcagacaacctgcagtgtgttggagcaccatcgagacctgatgaagagggtgaccggctgcatcctctccaacgtcaacacaccttccatgacacctgtgatcggacagccacagcatgagaacacccagcccatttaccagaacagcaatgtgcccaccaagcccactgccgcggagaacgccaacctgggcacagtgatgcggtatgaggagatcgagctgcgcatcttgctgctgtgtggtagtgacctgctggagtctttctgcatcccaggactctggaatgaggcagatatggaagtgattgttggggactttgggatcgtcgtggtacccagggatgcagcggacacagaccggatcatgaatcactcctccatactccgcaagtacaaaaacaacatcatggttgtgaaggatgatatcaaccatcccatgtctgtagtcagctccaccaagagcaggctggccctgcagcatggggacggccatgttgtggattacctgtcccagccagtcatcgattacatcctcaagagtcagctgtacatcaacgcctcgggctag

SEQ ID NO: 10 - protein, 280 aa, Mus musculus
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQHENTQPIYQNSNVPTKPTAAENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 11 - DNA, 6939 bp, synthetic construct
  source 1..6939: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatttacgtagccatgctctaggtaccattgacgtcaataatgacgtatgttcccatagtaacgccaatagggactttccattgacgtcaatgggtggagtatttacggtaaactgcccacttggcagtacatcaagtgtatcatatggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaaggaattgaattggatccgccaccatgtacccatacgatgttcctgactatgcgggctatccgtatgacgtcccggactatgcagggtcctatccatatgacgttccagattacgctagatctgaattgatgaccgagaccacaaagacccacgttatcctgctggcctgcggcagcttcaatcccatcactaaagggcacattcagatgttcgagagagccagggattatctgcacaagactggaagatttattgtgattggcgggattgtctctccggtccatgactcctacggaaaacagggccttgtgtcaagtcggcaccgtctcatcatgtgtcagctggctgtccagaattccgactggatcagggtggacccatgggagtgctatcaggacacctggcagacaacctgcagtgtgttggagcaccatcgagacctgatgaagagggtgaccggctgcatcctctccaacgtcaacacaccttccatgacacctgtgatcggacagccacagcatgagaacacccagcccatttaccagaacagcaatgtgcccaccaagcccactgccgcggagaacgccaacctgggcacagtgatgcggtatgaggagatcgagctgcgcatcttgctgctgtgtggtagtgacctgctggagtctttctgcatcccaggactctggaatgaggcagatatggaagtgattgttggggactttgggatcgtcgtggtacccagggatgcagcggacacagaccggatcatgaatcactcctccatactccgcaagtacaaaaacaacatcatggttgtgaaggatgatatcaaccatcccatgtctgtagtcagctccaccaagagcaggctggccctgcagcatggggacggccatgttgtggattacctgtcccagccagtcatcgattacatcctcaagagtcagctgtacatcaacgcctcgggctagacgcgtctcgagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 12 - DNA, 924 bp, Homo sapiens
  source 1..924: mol_type = other DNA
atgacagagaccaccaagacccatgtgatcctgctggcctgtggcagcttcaaccccatcaccaagggccacatccagatgtttgaaagagccagagactacctgcacaagacaggcagattcattgtgattggaggcatagtgagccctgtgcatgactcctatggcaagcaaggcctggtgagcagcagacacagactgatcatgtgccaactggctgtccagaactctgactggatcagggtggacccttgggaatgctaccaggacacctggcagaccacctgctctgtcctggaacaccacagagacctgatgaagagggtgacaggctgcatcctgagcaatgtgaacacccctagcatgacccctgtgattggacagcctcagaatgagacccctcagcccatctaccagaacagcaatgtggccaccaagcctacagctgccaagatcctgggcaaagtgggagagagccttagcaggatctgctgtgtgaggcccccagttgagagattcacatttgtggatgagaatgccaacctgggcacagtgatgagatatgaggagattgaactgagaatcctgctcctgtgtggctctgacctgctggagtccttctgcatccctggcctgtggaatgaggctgacatggaagtgattgtgggagactttggcatagtggtggtccctagagatgctgcagacacagatagaatcatgaaccacagctccatcctgagaaaatacaagaacaacatcatggtggtgaaggatgacatcaaccaccccatgtctgtggtgagcagcaccaagagcagactggccctgcagcatggtgatggccatgtggtggactacctgagccagccagtgatagattacatcctgaaaagccagctgtacatcaatgcctctggctga

SEQ ID NO: 13 - DNA, 843 bp, Homo sapiens
  source 1..843: mol_type = other DNA
atgacagagaccaccaagacccatgtgatcctgctggcctgtggcagcttcaaccccatcaccaagggccacatccagatgtttgaaagagccagagactacctgcacaagacaggcagattcattgtgattggaggcatagtgagccctgtgcatgactcctatggcaagcaaggcctggtgagcagcagacacagactgatcatgtgccaactggctgtccagaactctgactggatcagggtggacccttgggaatgctaccaggacacctggcagaccacctgctctgtcctggaacaccacagagacctgatgaagagggtgacaggctgcatcctgagcaatgtgaacacccctagcatgacccctgtgattggacagcctcagaatgagacccctcagcccatctaccagaacagcaatgtggccaccaagcctacagctgccgagaatgccaacctgggcacagtgatgagatatgaggagattgaactgagaatcctgctcctgtgtggctctgacctgctggagtccttctgcatccctggcctgtggaatgaggctgacatggaagtgattgtgggagactttggcatagtggtggtccctagagatgctgcagacacagatagaatcatgaaccacagctccatcctgagaaaatacaagaacaacatcatggtggtgaaggatgacatcaaccaccccatgtctgtggtgagcagcaccaagagcagactggccctgcagcatggtgatggccatgtggtggactacctgagccagccagtgatagattacatcctgaaaagccagctgtacatcaatgcctctggctga

SEQ ID NO: 14 - DNA, 6680 bp, synthetic construct
  source 1..6680: mol_type = other DNA
ctgcgcgctcgctcgctcactgaggccgcccgggcaaagcccgggcgtcgggcgacctttggtcgcccggcctcagtgagcgagcgagcgcgcagagagggagtgccaactccatcactaggggttccttgtagttaatgattaacccgccatgctacttatttacgtagccatgctctaggtaccattgacgtcaataatgacgtatgttcccatagtaacgccaatagggactttccattgacgtcaatgggtggagtatttacggtaaactgcccacttggcagtacatcaagtgtatcatatggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaaggaattgaattggatccgccaccatgacagagaccaccaagacccatgtgatcctgctggcctgtggcagcttcaaccccatcaccaagggccacatccagatgtttgaaagagccagagactacctgcacaagacaggcagattcattgtgattggaggcatagtgagccctgtgcatgactcctatggcaagcaaggcctggtgagcagcagacacagactgatcatgtgccaactggctgtccagaactctgactggatcagggtggacccttgggaatgctaccaggacacctggcagaccacctgctctgtcctggaacaccacagagacctgatgaagagggtgacaggctgcatcctgagcaatgtgaacacccctagcatgacccctgtgattggacagcctcagaatgagacccctcagcccatctaccagaacagcaatgtggccaccaagcctacagctgccaagatcctgggcaaagtgggagagagccttagcaggatctgctgtgtgaggcccccagttgagagattcacatttgtggatgagaatgccaacctgggcacagtgatgagatatgaggagattgaactgagaatcctgctcctgtgtggctctgacctgctggagtccttctgcatccctggcctgtggaatgaggctgacatggaagtgattgtgggagactttggcatagtggtggtccctagagatgctgcagacacagatagaatcatgaaccacagctccatcctgagaaaatacaagaacaacatcatggtggtgaaggatgacatcaaccaccccatgtctgtggtgagcagcaccaagagcagactggccctgcagcatggtgatggccatgtggtggactacctgagccagccagtgatagattacatcctgaaaagccagctgtacatcaatgcctctggctgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaaatctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaggttaattaaggcacacccgccgcgcttaatgcgccgctacagggcgcgtcccattcgccattcaggctgcgcaactgttgggaagggcgatcggtgcgg

SEQ ID NO: 15 - DNA, 6599 bp, synthetic construct
  source 1..6599: mol_type = other DNA
ctgcgcgctcgctcgctcactgaggccgcccgggcaaagcccgggcgtcgggcgacctttggtcgcccggcctcagtgagcgagcgagcgcgcagagagggagtgccaactccatcactaggggttccttgtagttaatgattaacccgccatgctacttatttacgtagccatgctctaggtaccattgacgtcaataatgacgtatgttcccatagtaacgccaatagggactttccattgacgtcaatgggtggagtatttacggtaaactgcccacttggcagtacatcaagtgtatcatatggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaaggaattgaattggatccgccaccatgacagagaccaccaagacccatgtgatcctgctggcctgtggcagcttcaaccccatcaccaagggccacatccagatgtttgaaagagccagagactacctgcacaagacaggcagattcattgtgattggaggcatagtgagccctgtgcatgactcctatggcaagcaaggcctggtgagcagcagacacagactgatcatgtgccaactggctgtccagaactctgactggatcagggtggacccttgggaatgctaccaggacacctggcagaccacctgctctgtcctggaacaccacagagacctgatgaagagggtgacaggctgcatcctgagcaatgtgaacacccctagcatgacccctgtgattggacagcctcagaatgagacccctcagcccatctaccagaacagcaatgtggccaccaagcctacagctgccgagaatgccaacctgggcacagtgatgagatatgaggagattgaactgagaatcctgctcctgtgtggctctgacctgctggagtccttctgcatccctggcctgtggaatgaggctgacatggaagtgattgtgggagactttggcatagtggtggtccctagagatgctgcagacacagatagaatcatgaaccacagctccatcctgagaaaatacaagaacaacatcatggtggtgaaggatgacatcaaccaccccatgtctgtggtgagcagcaccaagagcagactggccctgcagcatggtgatggccatgtggtggactacctgagccagccagtgatagattacatcctgaaaagccagctgtacatcaatgcctctggctgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaaatctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaggttaattaaggcacacccgccgcgcttaatgcgccgctacagggcgcgtcccattcgccattcaggctgcgcaactgttgggaagggcgatcggtgcgg

(19) United States
(12) Patent Application Publication
(10) Pub. No.: US 2024/0293581 A1
(43) Pub. Date: Sep. 5, 2024

(54) NEUROPROTECTION GENE THERAPY

(71) Applicant: The Board of Trustees of the Leland Stanford Junior University, Stanford, CA (US)

(72) Inventor: Yang Hu, Menlo Park, CA (US)

(21) Appl. No.: 18/659,959

(22) Filed: May 9, 2024

Related U.S. Application Data
(63) Continuation-in-part of application No. PCT/US2022/079396, filed on Nov. 7, 2022.
(60) Provisional application No. 63/277,982, filed on Nov. 10, 2021.

(51) Int. Cl. A61K 48/00; A61K 38/45; A61P 25/28; A61P 27/06; C12N 15/86

(52) U.S. Cl. CPC A61K 48/0058; A61K 38/45; A61K 48/0066; A61K 48/0075; A61P 25/28; A61P 27/06; C12N 15/86; C12Y 207/07001; C12N 2750/14143

(57) ABSTRACT

Compositions and methods for treating a mammalian subject for an axonopathy, including an optic nerve axonopathy, e.g. glaucoma. Aspects of the composition include a mammalian viral vector, comprising a γ-synuclein promoter, or functional fragment thereof, that promotes expression of a NMNTA2 transgene specifically in retinal ganglion cells (RGCs). Aspects of the methods include intravitreally administering the composition to treat the subject for an ON neuropathy.

### CROSS-REFERENCE

This application is a continuation in part of and claims the benefit of PCT Application No. PCT/US2022/079396, filed Nov. 7, 2022, which claims the benefit of U.S. Provisional Patent Application No. 63/277,982, filed Nov. 10, 2021, which the application is incorporated herein by reference in its entirety.

### STATEMENT REGARDING FEDERALLY SPONSORED RESEARCH

This invention was made with Government support under contract EY023295 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a Sequence Listing XML, “S21-363_STAN-1899WO_SeqList” created on May 9, 2024, and having a size of 42,500 bytes. The contents of the Sequence Listing XML are incorporated by reference herein in their entirety.

### INTRODUCTION

When neurons' axons are damaged by trauma, injury or disease, an axon degeneration process called Wallerian degeneration (WD) can be initiated, where neuron loss is preceded by degeneration of the neuronal axon, which is more pronounced at the distal end of axonal processes. WD is relevant to both the peripheral and central nervous systems and has been implicated in neurodegeneration diseases and conditions associated with neurological trauma.

NAD is synthesized via de novo production from tryptophan, and salvage pathways from nicotinamide (NAM), nicotinic acid (NA) or nicotinamide riboside (NR). NMNAT (nicotinamide mononucleotide adenylyl transferase) is essential for NAD production in both de novo and salvage pathways. There are three NMNATs (NMNAT1-3), NMNAT1 is localized to the nucleus and NMNAT3 is located in mitochondria; only NMNAT2 is enriched in neurons, especially in axons. Increased activity of NMNAT2 (nicotinamide mononucleotide adenylyl transferase 2) in nerve cells appears to provide neuronal protection by maintaining cellular levels of the metabolic cofactor nicotinamide adenine dinucleotide (NAD). Nerve injury causes a rapid decline in cellular NMNAT2 levels and a subsequent fall in NAD levels, followed by cellular bioenergetic crisis and axon degeneration and neuronal cell death.

Glaucoma is the most common cause of irreversible blindness and will affect more than 100 million people worldwide between 40 to 80 years old by 2040. It is characterized by optic neuropathy with optic nerve (ON) degeneration followed by progressive retinal ganglion cell (RGC) death. Although glaucoma can occur at any intraocular pressure (IOP) level, elevated IOP is associated with accelerated progression, probably due to mechanical damage of the ON head. The only available treatments act by reducing IOP, but fail to completely prevent the progression of glaucomatous neurodegeneration, indicating the urgent need for neuroprotection therapies.

Novel neuroprotective treatments are desperately needed. The lack of neuroprotective treatments for retinal ganglion cells (RGC) and optic nerve is a central challenge for glaucoma management. The only available treatments act by reducing intraocular pressure, but fail to completely prevent the progression of glaucomatous neurodegeneration, indicating the urgent need for neuroprotection therapies.

The instant disclosure provides novel compositions and methods of treatment for optic neuropathies including glaucoma.

### SUMMARY

Compositions and methods are provided for the treatment of a mammalian subject for an axonopathy. Compositions of the disclosure include a therapeutic gene therapy vector encoding an NMNAT2 coding sequence operably linked to a neuron-specific promoter, which may be referred to herein as a therapeutic NMNAT2 vector; polynucleotide constructs and cells for producing such a therapeutic NMNAT2 vector, and virus particles comprising such a therapeutic NMNAT2 vector. The vector of any of SEQ ID NO:11, SEQ ID NO:14 and SEQ ID NO:15 are provided as non-limiting examples of suitable vectors.

In some embodiments the NMNAT2 coding sequence is a human NMNAT2 coding sequence. In some embodiments the human NMNAT2 coding sequence encodes a variant protein with extended half-life relative to the wild-type protein, e.g. comprising a deletion of exon 6. In some embodiments the human NMNAT2 coding sequence is a codon-optimized sequence, e.g. SEQ ID NO:12 or SEQ ID NO:13. In some embodiments the vector is an adeno-associated virus or AAV vector. In some embodiments a virus particle comprising a therapeutic NMNAT2 vector is an adeno-associated virus (AAV). In some embodiments the neuron-specific promoter is selectively expressed in retinal ganglion cells (RGCs). In some embodiments the promoter is a γ-synuclein promoter (Sncg).

Methods are provided for reducing both neuronal cell body and axon death that results from axonopathies, the methods comprising contacting a neuron with an effective dose of a therapeutic NMNAT2 vector disclosed herein. The contacting may be performed in vivo, e.g. on a human subject. In some embodiments the therapeutic NMNAT2 vector is administered as a virus particle formulation. In some embodiments the formulation is administered to an individual intravitreally, for retina targeting. In some such embodiments the individual suffers from, or is at risk of developing, an optic nerve neuropathy, including without limitation, glaucoma.

In some embodiments a therapeutic formulation is provided, comprising a therapeutic NMNAT2 vector of the disclosure and a physiologically acceptable excipient. In some embodiments the vector is an AAV vector, which may be provided as a virus-particle. In some embodiments a virus particle comprising a therapeutic NMNAT2 vector is an adenovirus-associated virus. In some embodiments the therapeutic formulation is provided in a unit dose, where a unit dose may comprise from about 10⁹ to about 10¹⁵ vector genomes/eye of the therapeutic NMNAT2 vector. The therapeutic formulation may be provided in a kit further comprising components for intravitreal administration, e.g. microcapillary needles, diluents, and the like.

Conditions for treatment include central and peripheral nervous systems axonopathies, particularly conditions involving Wallerian degradation. The axonopathy may be the result of disease or trauma, including for example: CNS axonopathies such as amyotrophic lateral sclerosis (ALS) and hereditary spastic paraplegia (HSP); PNS nerve injury; traumatic brain injury; spinal cord injury or neuronal injury induced by a toxic agent such as a chemotherapeutic agent; and the like. In an embodiment, the axonopathy is a neuronal injury induced by a chemotherapeutic agent, e.g. a taxane, vincristine, etc. In some embodiments the axonopathy is an optic nerve neuropathy. In some embodiments the optic nerve neuropathy is glaucoma, e.g. open-angle glaucoma, angle-closure glaucoma, etc. In other embodiments an optic neuropathy is non-arteritic ischemic optic neuropathy (NAION), optic neuritis, ischemic optic neuropathy, inflammatory (non-demyelinating) and traumatic optic neuropathy, etc.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1E. RGC-specific transcriptome profiling in glaucoma reveals downregulation of NMNAT2. (A) Images of retinal sections and wholemounts showing co-localization of the Ribo-tag (HA-Rpl22) labeled by HA antibody and RGCs labeled by RBPMS antibody. Scale bar of retinal section, 20 μm; whole mount retina, 50 μm. (B) Heatmap of differentially expressed genes (DEGs) comparing glaucomatous RGCs to naïve RGCs. Triplicate samples from each group. (C) Gene ontology (GO) enrichment analysis of DEGs. Bar plot of the top 10 GO-enriched biological processes of the DEGs in glaucomatous RGCs. (D) GO enrichment analysis of DEGs in nicotinamide nucleotide biosynthetic and metabolic processes. (E) The expression levels and fold changes in glaucomatous and naïve RGCs of individual genes involved in NAD₊ biosynthesis and metabolism.

FIGS. 2A-2B. Both NMNAT2 and NMNAT1 overexpression promote neuroprotection of RGC somata and axons after ON crush injury. (A) Upper panel, confocal images of peripheral flat-mounted retinas showing surviving RBPMS positive (red) RGCs 2 weeks post crush injury. Scale bar, 20 μm. Lower panel, light microscope images of semi-thin transverse sections of ON with PPD staining 2 weeks post crush injury. Scale bar, 10 μm. (B) Quantification of surviving RGC somata and axons, represented as percentage of crush injured eyes compared to the sham contralateral control eyes. Data are presented as means±s.e.m, n=10 of each group; *: p<0.05, ****: p<0.0001, one-way ANOVA with Tukey's multiple comparisons test.

FIGS. 3A-3E. RGC-specific NMNAT2 overexpression significantly promotes neuronal NAD⁺ production and survival of both RGC somata and axons in SOHU glaucoma model. (A) Relative NAD⁺ levels of retinas and ONs from naïve, SOHU glaucoma at 1 week post SO injection (1 wpi), and SOHU glaucoma mice with NMNAT1 or NMNAT2 overexpression, acquired by LC/MS analysis. n=5 in all the groups. Data are presented as means±SD, *: p<0.05, **: p<0.01, one-way ANOVA with Dunnett's multiple comparisons test. (B) Representative OCT images of SOHU glaucoma mouse retinas at 3 wpi. GCC: ganglion cell complex, including RNFL, GCL and IPL layers; indicated as double end arrows. (C) Quantification of GCC thickness measured by OCT at 3 wpi, represented as percentage of GCC thickness in the SOHU eyes compared to the sham contralateral control eyes. n=20 in both groups. Data are presented as means±SD, ***: p<0.001, two-tailed unpaired t test. (D) Upper panel, representative confocal images of peripheral flat-mounted retinas showing surviving RBPMS positive (red) RGCs at 3 wpi. Scale bar, 20 μm. Lower panel, light microscope images of semi-thin transverse sections of ON with PPD staining at 3 wpi. Scale bar, 10 μm. (E) Quantification of surviving RGCs somata and axons at 3 wpi, represented as percentage of glaucomatous eyes compared to the sham contralateral control eyes. n=19 in both groups. Data are presented as means±SD, ****: p<0.0001, two-tailed unpaired t test.

FIGS. 4A-4B. NMNAT2 overexpression preserves visual functions of glaucomatous mice. (A) Left: representative wave forms of PERG at 3 wpi. Right: quantification of P1-N2 amplitude of PERG at 3 wpi, represented as percentage of glaucomatous eyes compared to the sham contralateral control eyes. n=20 in both groups. Data are presented as means±s.e.m, ****: p<0.0001, two-tailed unpaired t test. (B) Visual acuity measured by OKR at 3 wpi, represented as percentage of glaucomatous eyes compared to the sham contralateral control eyes. Data are presented as means±s.e.m, ****: p<0.0001, two-tailed unpaired t test.

FIG. 5. Map of therapeutic AAV-mSncg-NMNAT2 vector.

FIGS. 6A-6E. AAV2-mSncg-mediated RGC-specific expression of NMNAT2Δex6 after intravitreal injection. (A) Representative confocal images of retina wholemounts showing RBPMS positive (green) RGCs and HA-tagged NMNAT2 (red) overexpression in mice 2 weeks after intravitreal injection of AAV2-mSncg-3HA-NMNAT2Δex6, but not in mice injected with AAV2-mSncg-control. Scale bar, 20 μm. (B) Representative confocal images of retina cross-sections showing HA-tagged NMNAT2 (red) expression in RBPMS positive (green) RGCs but not in other layers of retina, 2 weeks after intravitreal injection. GCL: ganglion cell layer. Scale bar, 20 μm. (C) The NAD₊ level in retinas with NMNAT2 overexpression is significantly higher than in control retinas. Data are presented as means±s.e.m, n=4 of each group; **: p<0.01, two-tailed unpaired t test. (D) Representative confocal images of ON longitudinal sections immunostained for Tuj1 and HA in mice 2 weeks after intravitreal injection. Scale bar, 20 μm. (E) The NAD₊ levels are significantly greater in ONs with NMNAT2 overexpression than control ONs. Data are presented as means±s.e.m, n=4 of each group; **: p<0.01, two-tailed unpaired t test.

FIGS. 7A-7B. NMNAT2 overexpression does not affect IOP elevation but protects RGCs significantly in glaucomatous mice. (A) IOP measurements at 3 wpi. Naïve, n=20 of all the groups. Data are presented as means±s.e.m, ****: p<0.0001, one-way ANOVA with Tukey's multiple comparisons test. (B) Representative fluorescence microscope images of the whole flat-mounted retinas showing surviving RBPMS positive (red) RGCs at 3 wpi. Scale bar, 500 μm.

FIGS. 8A-8B. Map of therapeutic AAV-mSncg-NMNAT2 vector with (A) codon optimized cophNMNAT2 sequence. The vector sequence is provided as SEQ ID NO:14, the codon-optimized NMNAT2 sequence is provided as SEQ ID NO: 12. (B) Schematic of a vector with codon optimized Δexon6 (cophNMNAT2ΔEx6) sequence. The vector sequence is provided as SEQ ID NO:15, the codon-optimized NMNAT2ΔEx6 sequence is provided as SEQ ID NO: 13.

### DETAILED DESCRIPTION OF THE EMBODIMENTS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference in their entirety, as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compositions and methods have been or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

AAV Vectors. Utilizing a viral vehicle to deliver genetic material into cells allows direct targeting of pathogenic molecules and restoration of function. The retina is an advantageous target for gene therapy due to its easy access, confined non-systemic localization, partial immune privilege, and well-established definitive functional readouts. The success of adeno-associated virus (AAV)-mediated gene replacement in treating inherited retinal disease makes RGC-specific therapy with AAV a promising gene therapy strategy for optic neuropathies. Because AAV is non-pathogenic and cannot reproduce itself without helper viruses, it has served as a primary vehicle for gene therapy. It is a single-stranded DNA virus that stably and efficiently infects a wide variety of cells in multiple tissues. AAV2, the best-characterized AAV serotype, efficiently infects RGCs in retina after intravitreal injection, and may be used in the disclosed methods.

In some embodiments, a vector for the present disclosure is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vectors exist in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Rudder Arnold, London, UK (2006); and D E Bowles, J E Rabinowitz, R J Samulski “The Genus Dependovirus” (J R Kerr, SF Cotmore. M E Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and W0/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAVIO, AAVII, AAV12, AAV13, AAV14, AAV15, and AAV16). In some embodiments the vector is AAV2. Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

Neuron-specific Promoter. A neuron-specific promoter allows precise manipulation of gene expression from a vector, without affecting other cell types. Aspects of the present invention encompass expression cassettes and/or vectors comprising polynucleotide sequences of interest for expression in targeted cells. The polynucleotides can comprise promoters operably linked to an NMNAT2 coding sequence. Targeted expression is accomplished using a cell-selective or cell-specific promoter. Examples are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Neuron-specific promoters of particular interest include RGC specific promoters, e.g. murine γ-synuclein (mSncg) promoter, which drives specific, potent and sustained transgene expression in rodent RGCs, nonhuman primate RGCs, and human primary RGCs, as well as human induced Pluripotent Stem Cell (IPS) stem cell-derived RGCs. See, for example, Wang et al. (2020) J Neurosci 40(20):3896-3914, herein specifically incorporated by reference.

In some embodiments a neuron-specific promoter is used for the selective expression of an operably linked gene in retinal ganglion cells (RGCs). In some embodiments the promoter comprises or consists of an mSncg promoter, optionally selected from the sequence set forth in SEQ ID NO:1, 2, 3, or 4, or a sequence having at least 95% sequence identity to a sequence selected from SEQ ID NO:1, 2, 3, or 4. In some embodiments the promoter sequence is provided in the context of a vector for expression, including without limitation a viral vector, e.g. an AAV vector, and is operably linked to a sequence desired for expression, e.g. human NMNAT2. Cells of interest for expression include, without limitation, neuronal cells in the eye and progenitors thereof, e.g. retinal cells, particularly retinal ganglion cells, and their progenitors.

NMNAT (Nicotinamide/nicotinate mononucleotide adenylyltransferase) is the central enzyme of the NAD⁺ (nicotinamide adenine dinucleotide) biosynthetic pathway, catalyzing the formation of NAD⁺ from NMN⁺ (nicotinamide mononucleotide) and NaAD (nicotinic acid adenine dinucleotide) from NaMN (nicotinic acid mononucleotide). Three isoforms of the enzyme have been identified, expressed by three different genes in mammals: NMNAT1, NMNAT2 and NMNAT3. NMNAT2 exists in more than one splice form. It is mainly expressed in brain, heart and muscle tissue. At the cellular level, NMNAT2 is abundant in the Golgi complex. NMNAT2 is known to be broken down via the ubiquitin-proteasome system whereby proteins destined for destruction undergo molecular tagging with ubiquitin which targets them for proteasomal breakdown. The enzymes that catalyze the ubiquitination step are called ubiquitin ligases. Preventing or slowing the reaction that results in the degradation of Nmnat2 represents an attractive target mode of action for an NMNAT2 vector.

In one embodiment, an NMNAT2 therapeutic vector functions to provide a human NMNAT2 protein, e.g. having the protein sequence of any of NP_055854.1, NP_733820.1, XP_024310045.1, including, for example, SEQ ID NO:5 or a variant thereof. A codon-optimized sequence encoding SEQ ID NO:5 is provided as SEQ ID NO:12.

In some embodiments a human NMNAT2 protein in a therapeutic vector disclosed herein has an extended half-life relative to the wild-type protein. Wild-type NMNAT2 is rapidly cleared from the cell, with a half-life of from about 2-3 hours in cultured cells. The constant supply and degradation of the protein ensures that a neuron is highly responsive to environmental or cellular changes and can initiate a cascade leading to axonal degeneration within a matter of hours if necessary. In some embodiments an extended half-life variant comprises a deletion of exon 6, which may be denoted herein as NMNAT1Δexon 6, for example as set forth in SEQ ID NO:6. A codon-optimized sequence encoding SEQ ID NO:6 is provided as SEQ ID NO:13. The increase in half-life may be up to about 2-fold, up to about 3-fold, up to about 4-fold, up to about 5-fold, up to about 10-fold, up to about 20-fold, up to about 50-fold, up to about 100-fold, or more.

In some embodiments an NMNAT2 sequence comprises at least about 90% sequence identity, at least 95% sequence identity, at least about 97%, sequence identity, at least about 99% sequence identity to a reference sequence of any of the above-disclosed human NMNAT2 proteins. In some embodiments a codon-optimized DNA sequence is provided, encoding full-length or truncated (exon 6 deleted) NMNAT2, as SEQ ID NO:12 or SEQ ID NO:13, respectively.

As used herein, the term “wildtype” generally refers to a gene, or sub-portion thereof, in the subject that is not mutated, or not substantially mutated (e.g., at either allele) so as to affect the function of the gene. Accordingly, a wildtype locus may contain the common (i.e., most prevalent, normal, etc.) sequence of the gene, or essentially the common sequence of the gene, without mutation, or without substantial mutation, affecting the function of the gene. The “common sequence”, as used in this context, generally refers to the gene sequence as it most frequently occurs in a natural population. In some instances, common sequences may be represented by a reference sequence, e.g., a reference sequence as it appears in a sequence database, such as but not limited to e.g., GenBank database (NCBI), UniProt database (EBI/SIB/PIR), or the like. In some instances, a wildtype locus may be identical or substantially identical to a reference sequence.

By “treatment” it is meant that at least an amelioration of one or more symptoms associated with a neurodegenerative disorder afflicting the subject is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs (e.g., the optic nerve (ON)), etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The term ‘neuroprotective’ as used herein refers to the ability to protect neurons or their axons or synapses in the central or peripheral nervous system from damage or death. Many different types of insult can lead to neuronal damage or death, for example: metabolic stress caused by hypoxia, hypoglycemia, diabetes, loss of ionic homeostasis or other deleterious process, physical injury of neurons, exposure to toxic agents and numerous diseases affecting the nervous system including inherited disorders. The presence of an agent that is neuroprotective enables a neuron to remain viable upon exposure to insults that would otherwise cause a loss of functional integrity in an unprotected neuron.

Axonopathy is broadly defined as functional or structural defects in the axon or its terminal, and has been established as a major early contributor to the genesis, progression, and symptomology of neurodegenerative disorders. Axon degeneration is an active process, as demonstrated in Wallerian degeneration, which involves the fragmentation and disintegration of an axon distal to the site of an injury. Axonopathy is often considered in the context of peripheral motor and sensory neurons, given their length, the presence of diseases that specifically affect these systems, and their sensitivity to challenges such as chemotherapy drugs or metabolic disorders such as diabetes. However, these characteristics are not limited to the peripheral nervous system.

Glaucoma, a neuropathy affecting axons of the optic nerve, one of the few central nervous system components outside of the brain and spinal cord. Glaucoma shares common features with other central neurodegenerations such as amyotrophic lateral sclerosis (ALS) and hereditary spastic paraplegia (HSP), Alzheimer's, Parkinson's, and Huntington's diseases, often exhibiting comorbidity with those conditions, as well as exhibiting similar mechanisms with these and other axonopathies.

NMNAT2 therapeutic vectors are of utility in the treatment of neurodegenerative disorders involving Wallerian degeneration. Examples of disorders where such degeneration can be of importance include Alexander's disease, Alper's disease, Alzheimer's disease, Amyotrophic lateral sclerosis, Ataxia telangiectasia, Batten disease, Canavan disease, Cerebral palsy, Cockayne syndrome, Corticobasal degeneration, Creutzfeldt-Jakob disease, Diabetic neuropathy, Frontotemporal lobar degeneration, Glaucoma, Guillain-Barre syndrome, Hereditary spastic paraplegia, Huntington's disease, HIV associated dementia, Kennedy's disease, Krabbe's disease, Lewy body dementia, Motor neuron disease, Multiple System Atrophy, Multiple sclerosis, Narcolepsy, Neuroborreliosis, Niemann Pick disease, Parkinson's disease, Pelizaeus-Merzbacher Disease, Peripheral neuropathy, Pick's disease, Primary lateral sclerosis, Prion diseases, Progressive Supranuclear Palsy, Refsum's disease, Sandhoff's disease, Schilder's disease, Spinocerebellar ataxia, Spinal cord injury, Spinal muscular atrophy, Steele-Richardson-Olszewski disease, Stroke and other ischaemic disorders, Tabes dorsalis or Traumatic brain injury.

In an embodiment a therapeutic vector of the disclosure is used as a neuroprotective medicament in the treatment of a neurodegenerative disorder resulting from neuronal injury. In a further embodiment the therapeutic vector is used as a neuroprotective medicament in the treatment of a neurodegenerative disorder involving Wallerian degeneration resulting from neuronal injury. In one embodiment, the neuronal injury results from trauma. In one embodiment, the neuronal injury is induced by a chemotherapeutic agent.

Certain drugs used in cancer chemotherapy, for example Taxol or vincristine, cause peripheral neuropathy, which limits the maximum doses at which they can be used. Recent studies show that neurons suffering from Taxol or vincristine toxicity undergo Wallerian-like changes in their morphology and in the underlying molecular events. Inhibiting Wallerian degeneration can be particularly effective in this condition as neurons are only temporarily exposed to the neurotoxic agent. Simultaneous administration of Taxol with an agent inhibiting Wallerian degeneration can therefore allow the drug to be used at substantially higher doses than is currently possible.

The term “injury” as used herein refers to damage inflicted on a neuron, whether in the cell body, or in axonal or dendritic processes. This can be a physical injury in the conventional sense i.e. traumatic injury to the brain, spinal cord or peripheral nerves caused by an external force applied to a subject. Other damaging external factors are for example environmental toxins such as mercury and other heavy metals, pesticides and solvents. Alternatively, injury can result from an insult to the neuron originating from within the subject, for example: reduced oxygen and energy supply as in ischemic stroke and diabetic neuropathy, autoimmune attack as in multiple sclerosis or oxidative stress and free-radical generation as is believed to be important in amyotrophic lateral sclerosis. Injury is also used here to refer to any defect in the mechanism of axonal transport.

In another embodiment, the therapeutic vector is intended for use as a neuroprotective medicament wherein the neurodegenerative disorder is caused by a neuronal injury resulting from a disease. In some instances, the optic neuropathy and/or neurodegenerative disorder treated according to the methods described herein may be an optic neuropathy such as Leber's hereditary optic neuropathy (LHON), Anterior ischemic optic neuropathy (AION), optic disc drusen (ODD), dominant optic atrophy (DOA), ON damage associated with glaucoma, or other CNS neurodegenerative disorder leading to ON degeneration. In some instances of the methods disclosed herein, the disease or disorder may involve inflammation leading to degeneration of the ON.

In one embodiment, the neurodegenerative disorder is an ophthalmic disorder such as glaucoma. In an embodiment a therapeutic vector of the disclosure is used as a neuroprotective medicament in the treatment of glaucoma. Glaucomas are a group of eye disorders characterized by progressive optic nerve damage in which an important part is a relative increase in intraocular pressure (IOP) that can lead to irreversible loss of vision. Glaucomas are categorized as open-angle glaucoma or angle-closure glaucoma. The “angle” refers to the angle formed by the junction of the iris and cornea at the periphery of the anterior chamber. The angle is where >98% of the aqueous humor exits the eye via either the trabecular meshwork and the Schlemm canal or the ciliary body face and choroidal vasculature. Glaucomas are further subdivided into primary (cause of outflow resistance or angle closure is unknown) and secondary (outflow resistance results from a known disorder), accounting for >20 adult types. Another group of glaucoma patients does not have IOP elevation, which in general is called normal tension glaucoma (NTG). NTG is also associated with progressive optic nerve degeneration and RGC death. Thus they are also subject to this gene therapy treatment.

Axons of retinal ganglion cells travel through the optic nerve carrying visual information from the eye to the brain. Damage to these axons causes ganglion cell death with resultant optic nerve atrophy and patchy vision loss. Elevated intraocular pressure (IOP; in unaffected eyes, the average range is 11 to 21 mm Hg) plays a role in axonal damage, either by direct nerve compression or diminution of blood flow. However, the relationship between externally measured pressure and nerve damage is complicated. Of people with IOP>21 mm Hg (ie, ocular hypertension), only about 1 to 2%/year (about 10% over 5 years) develop glaucoma. Additionally, about one third of patients with glaucoma do not have IOP>21 mm Hg (known as low-tension glaucoma or normal-tension glaucoma).

IOP is determined by the balance of aqueous secretion and drainage. Elevated IOP is caused by inhibited or obstructed outflow, not oversecretion; a combination of factors in the trabecular meshwork (eg, dysregulation of extracellular matrix, cytoskeletal abnormalities) appear to be involved. In open-angle glaucoma, IOP is elevated because outflow is inadequate despite an angle that appears unobstructed. In angle-closure glaucoma, IOP is elevated when a physical distortion of the peripheral iris mechanically blocks outflow.

Symptoms and signs of glaucoma vary with the type of glaucoma, but the defining characteristic is optic nerve damage as evidenced by an abnormal optic disk and certain types of visual field deficits. Glaucoma is diagnosed when characteristic findings of optic nerve damage are present and other causes have been excluded. Elevated IOP makes the diagnosis more likely, but elevated IOP can occur in the absence of glaucoma and is not essential for making the diagnosis.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) after the administration of a second therapeutic agent.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes lipoproteins, glycoproteins, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector, a guide RNA, a donor DNA template, and the like. For example, a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

### Methods

As summarized above, aspects of the instant disclosure include methods of treating a subject for an optic neuropathy. A variety of neurodegenerative disorders can be treated by practice of the methods described herein, particularly glaucoma, e.g. open-angle glaucoma or angle-closure glaucoma. In some embodiments, provided herein is a method of treating an optic nerve (ON) axonopathy in a mammalian subject in need thereof, comprising intravitreally administering the composition into the subject, thereby treating the ON axonopathy. In some embodiments, provided herein is a method of reducing or ameliorating degeneration of axons and/or soma of RGCs, comprising intravitreally administering the composition into a mammalian subject experiencing or at imminent risk of an ON axonopathy.

In some aspects, provided herein is a method of inducing neuroprotection/increasing survival/promoting functional recovery of RGC somata and axons, comprising intravitreally administering the composition into a mammalian subject experiencing or at risk of an ON axonopathy. In some embodiments of the method, the ON neuropathy is retinal ganglion cell degeneration, including glaucoma, optic neuritis, ON traumatic injury and other ON-related diseases.

In some embodiments, the therapeutic vector comprises an AAV vector, e.g. an AAV2 vector, that comprises a murine γ-synuclein promoter in operable linkage with a nucleic acid encoding a human or murine NMNAT2 protein. Non-limiting examples include the sequences set forth in any of SEQ ID NO:11, SEQ ID NO:14 and SEQ ID NO:15.

Various subjects may be treated in the methods of the present disclosure. In some instances, treated subjects may be mammals, including but not limited to e.g., rodents (e.g., rats, mice, etc.), non-human primates (e.g., macaques, marmosets, tamarins, spider monkeys, owl monkeys, vervet monkeys, squirrel monkeys, baboons, chimpanzees, etc.), humans, and the like. In some instances, a treated subject may be an animal model (e.g., a rodent model, a non-human primate model, etc.) of an optic neuropathy and/or neurodegenerative disorder.

In some instances, a treated subject is a human subject, including but not limited to e.g., a human subject having an optic neuropathy and/or neurodegenerative disorder, a human subject at increased risk of developing an optic neuropathy and/or neurodegenerative disorder, a human subject carrying an NMNAT2 mutation that is causative to a disease, a human subject with low NAD level in neurons, a human subject of advanced age (e.g., at least 60 years of age, at least 65 years of age, at least 70 years of age, at least 75 years of age, at least 80 years of age, at least 85 years of age, at least 90 years of age, etc.), or an individual having multiple such risk factors. Treated subjects may or may not be symptomatic, e.g., a subject may or may not display or have previously displayed one or more symptoms of an optic neuropathy and/or neurodegenerative disorder, including but not limited to e.g., those optic neuropathies and/or neurodegenerative disorders described herein.

Methods of the present disclosure may include administering to a subject a therapeutic NMNTA2 vector, e.g. in the form of a virus particle, that targets RGCs and reduces RGC and optic nerve degeneration; or a therapeutic NMNTA2 vector, e.g. in the form of a virus particle where the protein shares 100% sequence identity or less than 100% sequence identity, including e.g., at least 99%, at least 98%, at least 97% at least 96%, at least 95%, at least 90%, at least 85%, at least 80%, etc., sequence identity, with a protein or amino acid sequence of a protein described herein, e.g. a human NMNTA2 protein. In some instances, a vector comprises a polynucleotide encoding NMNTA2, or a fragment thereof, including where the polynucleotide shares 100% sequence identity or less than 100% sequence identity, including e.g., at least 99%, at least 98%, at least 97% at least 96%, at least 95%, at least 90%, at least 85%, at least 80%, etc., sequence identity, with an encoding polynucleotide identified herein. In some embodiments the vector comprises a sequence of any of SEQ ID NO:11, SEQ ID NO:14 or SEQ ID NO:15.

Polynucleotides of interest as promoters in the present disclosure include polynucleotide sequences having 100% sequence identity, or less than 100% sequence identity, including e.g., at least 99%, at least 98%, at least 97% at least 96%, at least 95%, at least 90%, at least 85%, at least 80%, etc., sequence identity, with a γ-synuclein promoter sequence as identified herein. In some instances, the a γ-synuclein promoter is from a mammal, e.g., a human or a rodent such as a mouse or rat. In some instances, the promoter has 100% sequence identity, or less than 100% sequence identity, including e.g., at least 99%, at least 98%, at least 97% at least 96%, at least 95%, at least 90%, at least 85%, or at least 80% sequence identity to the mSncg promoter or a fragment thereof.

In some instances, a subject treated according to the methods of the present disclosure have a mutation at one or more loci of target genes identified herein. Correspondingly, without limitation, a subject treated according to the methods of the present disclosure may have a mutation, e.g. a mutation causing reduced function of the encoded protein, at the NMNAT2 locus, CHOP locus, mutant at the SARM1 locus, mutant at the XBP-1 locus, mutant at the eIF2 locus, mutant at the ATF4 locus, mutant at the ATF6 locus, or mutant at a combination of loci thereof.

Administration of an agent to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to intravitreal injection, oral and parenteral routes, such as intravenous (iv), intraperitoneal (ip), rectal, topical, ophthalmic, nasal, and transdermal. Formulations for these dosage forms are described herein.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the subject, the amount or dosage is effective when administered for a suitable period of time, such as one week or longer, including two weeks or longer, such as 3 weeks or longer, 4 weeks or longer, 8 weeks or longer, etc., so as to evidence a reduction in the disorder, e.g., a reduction in a symptom of the disorder or in a marker of disease pathology. For example, an effective dose is the dose that, when administered for a suitable period of time, such as at least about one week, and maybe about two weeks, or more, up to a period of about 3 weeks, 4 weeks, 8 weeks, or longer, will reduce a symptom of the disorder, for example, by about 10% or more, by about 20% or more, e.g., by 30% or more, by 40% or more, or by 50% or more, in some instances by 60% or more, by 70% or more, by 80% or more, or by 90% or more, for example, and will halt progression of the disorder in the subject. In some instances, an effective amount or dose of active agent will not only slow or halt the progression of the disease condition but will also induce the reversal of the condition, i.e., will cause an improvement in the neurological health of the subject. For example, in some instances, an effective amount is the amount that when administered for a suitable period of time, for example, at least about one week, and/or about two weeks, or more, up to a period of about 3 weeks, 4 weeks, 8 weeks, or longer will improve, stabilize, or at least reduce the progression of a disorder in subject, for example 1.5-fold, 2-fold, 3-fold, 4-fold, 5-fold, in some instances 6-fold, 7-fold, 8-fold, 9-fold, or 10-fold or more relative to the subject's condition prior to administration.

In some instances, in those embodiments where an effective amount of an active agent is administered to the subject, the amount or dosage is effective when administered for a suitable period of time to result in a reduction in RGC degeneration in the subject. Such a reduction may manifest in various ways, including but not limited to e.g., an increase in the number, size or length of RGCs, or a reduction in the amount of degeneration of RGCs, or their axons or soma, or the like. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., reduction in RGC degeneration. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., increase in RGC number, size or length of RGC axons or somata. Various methods of assessing the amount of RGC degeneration or increase in number, size or length of RGC axons or somata may be employed, including invasive and non-invasive techniques, such as electrophysiology measurement for RGC neuronal function, visual acuity, OCT imaging, fundus imaging, histology studies of RGC somata and axons morphology.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent is an amount that is sufficient, when administered to the individual, to palliate, ameliorate, stabilize, reverse, prevent, slow or delay the progression of the disease state (e.g., neurodegeneration) by, for example, inhibiting gene expression product formation, or otherwise preventing the symptoms or clinical progression of a neurodegenerative disorder present in the subject.

In some embodiments a therapeutic dose is determined by the number of vector genomes administered to a retina, e.g. at least about 10⁸ vector genomes, at least about 10⁹, at least about 10¹⁰, and up to about 10¹⁵, up to about 10¹⁴, up to about 10¹², and may be from about 10⁸ to 10¹⁵, from about 10⁹ to about 10¹⁴, from about 10¹⁰ to about 10¹². The vector genomes may be administered in the form of virus particles. The volume in intravitreal injection, per injection, may be not more than about 500 μl, not more than about 200 μl, not more than about 100 μl, and may be from about 1 μl to about 200 μl, from about 5 μl to about 100 μl, from about 25 μl to about 100 μl, and may be around 50 μl.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, MD 20857, the disclosure of which is incorporated herein by reference).

### Pharmaceutical Compositions

A pharmaceutical composition comprising a therapeutic vector, e.g. an AAV virus particle comprising a therapeutic vector, may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, or any other dosage form suitable for administration.

A therapeutic vector may be administered to the host using any convenient means capable of resulting in the desired reduction in disease condition or symptom. Thus, a therapeutic vector can be incorporated into a variety of formulations for therapeutic administration. More particularly, a therapeutic vector can be formulated into pharmaceutical compositions by combination with appropriate pharmaceutically acceptable carriers or diluents, and may be formulated into preparations in solid, semi-solid, liquid or gaseous form.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed compositions may comprise a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A therapeutic vector can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A therapeutic vector can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, or other route of administration, e.g., injection into the retina.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

In some instances, methods of treating a subject as described herein may include administering to the subject an effective amount of an agent that reduces RGC degeneration in the subject.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of treating a neurodegenerative condition in a subject, including by administering to the subject an effective amount of a therapeutic vector that reduces the prevalence of RGC degeneration. The subject kits may include any combination of components (e.g., reagents, cell lines, etc.) for performing the subject methods, such as e.g., methods of treating a neurodegenerative condition and/or methods of identifying a RGC degeneration-associated target gene.

In some aspects, provided herein is a kit comprising an AAV vector, wherein the vector comprises a murine γ-synuclein promoter that promotes expression of a NMNTA2 coding sequence specifically in RGCs, wherein the murine γ-synuclein promoter is in operable linkage with an expression cassette; and instructions for use.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Example 1

### NMNAT2 is Downregulated in Glaucomatous RGCs and RGC-Specific Gene Therapy Rescues Neurodegeneration and Visual Function

The lack of neuroprotective treatments for retinal ganglion cells (RGCs) and optic nerve (ON) is a central challenge for glaucoma management. Emerging evidence suggests that redox factor NAD⁺ decline is a hallmark of aging and neurodegenerative diseases. Supplementation with NAD+ precursors and overexpression of NMNAT1, the key enzyme in the NAD+ biosynthetic process, have significant neuroprotective effects. Among the three NMNAT isoforms, only NMNAT2 is enriched in neurons, but its role in glaucoma is not known. Here we first present the translatomes profiled by RiboTag mRNA sequencing in both naïve and glaucomatous mouse RGCs. Intriguingly, only NMNAT2, but not NMNAT1 or NMNAT3, is significantly decreased in glaucomatous RGCs. We next demonstrate that AAV2-mediated overexpression of RGC-specific promoter mSncg-driven long half-life NMNAT2 mutant restores RGC NAD+ levels. And this gene therapy strategy delivers significant neuroprotection of both RGC soma and axon and preservation of visual function in the traumatic ON crush model and the ocular hypertension glaucoma model. Our studies show that the weakening of NMNAT2 expression in glaucomatous RGCs contributes to a deleterious NAD+ decline and that modulating RGC intrinsic NMNAT2 levels by AAV2-mSncg vector provides gene therapy for glaucomatous neurodegeneration.

It is believed that Wallerian degeneration of RGCs' axons plays a critical role in glaucomatous neurodegeneration and that Wallerian degeneration is closely associated with the axonal NAD₊ level; an adequate axonal NAD₊ level is both necessary and sufficient for axon survival. The chimeric mutant protein, slow Wallerian degeneration protein (Wlds), contains the full-length NAD₊-synthetic enzyme, nicotinamide mononucleotide adenylyltransferase 1 (NMNAT1), and part of the ubiquitin ligase UBE4B. It is translocated to axons, significantly delays Wallerian degeneration and rescues glaucomatous neurodegeneration. Cytoplasmic NMNAT1 mutant (cytNMNAT1) overexpression or feeding of the NAD₊ precursor, vitamin B3, has consistently been found to promote substantial RGC soma and axon protection in optic neuropathy models, indicating that modulating the neuronal NAD₊ level through upregulating NMNATs is a promising neuroprotective strategy for glaucoma. However, endogenous NMNAT1 is localized to the nucleus and NMNAT3 is located in mitochondria; only NMNAT2 is enriched in axons. This distribution suggests that NMNAT2 plays a predominant role in maintaining axonal integrity. Indeed, whereas NMNAT2 knockout mice have truncated ONs, NMNAT2 overexpression delays injury-induced axon degeneration both in vitro and in vivo. Therefore, NMNAT2 is a promising choice as a neuronal intrinsic neuroprotective target for axonopathies, but it has not been tested directly in glaucoma prior to this disclosure.

Taking advantage of our recently developed novel mouse glaucoma models and RGC-specific AAV promoter, we here first profiled the translatomes of naïve and glaucomatous RGCs. We found that NMNAT2 is the most abundant NMNAT in RGCs, and that NMNAT2, but not NMNAT1 or NMNAT3, is significantly decreased in glaucomatous RGCs prior to significant neurodegeneration. We then forced expression of a long-life NMNAT2 mutant by AAV intravitreal injection in RGCs and demonstrated that RGC-specific NMNAT2 overexpression has significant neuroprotective effects: it preserves RGC somata and axons and visual function in a clinically relevant mouse glaucoma model. This readily translatable gene therapy strategy contributes to developing efficient neuroprotective treatments for glaucoma by targeting neuronal intrinsic NMNAT2.

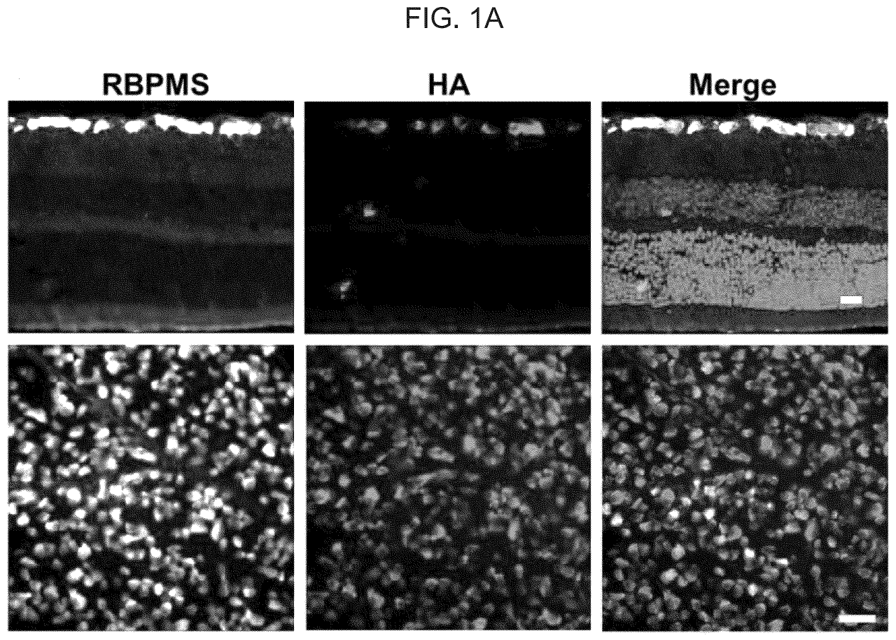

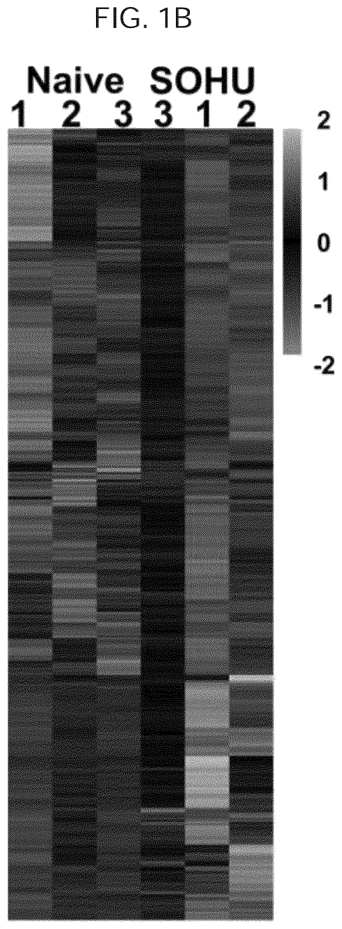

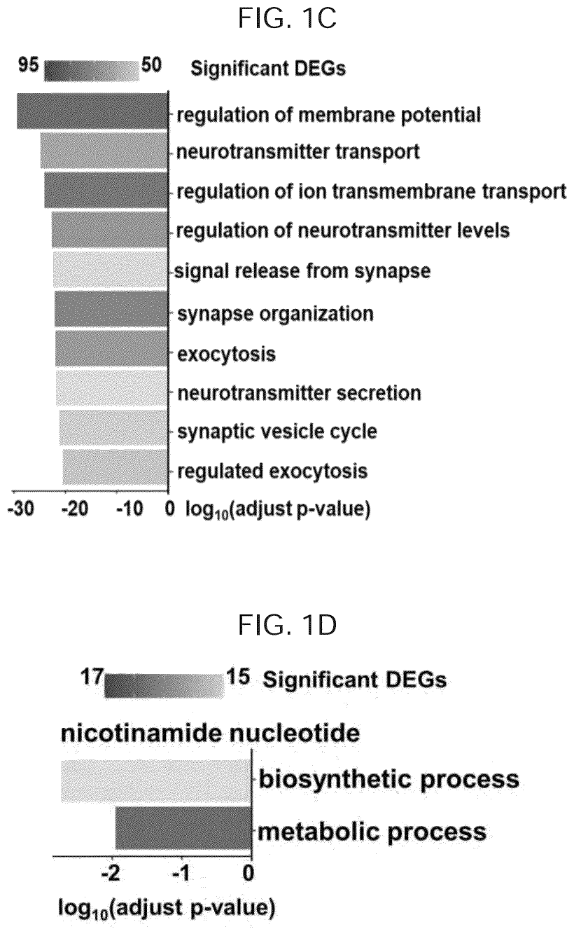

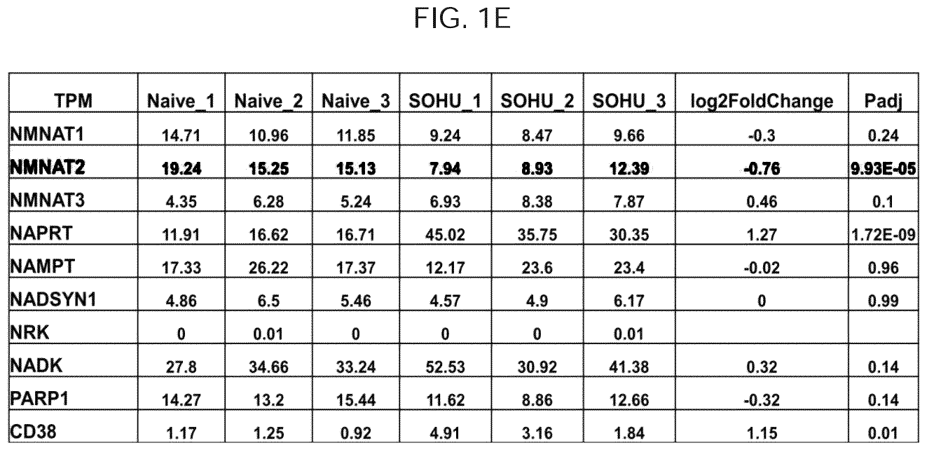

NMNAT2 is significantly decreased in glaucomatous RGCs. To profile RGC transcriptomes, we employed RiboTag mice, which were generated by knocking in the HA-tagged ribosome protein Rpl22 (HA-Rpl22) to the endogenous Rpl22 allele, immediately after the floxed endogenous Rpl22. Expression of HA-Rpl22 in RGCs specifically is achieved by intravitreal injection of AAV2-Cre driven by RGC-specific promoter mSncg and Cre-mediated deletion of endogenous Rpl22 in RGCs (FIG. 1A). We generated the SOHU glaucoma model in one eye and used the contralateral eye as naïve control. We then used HA antibody to immunoprecipitate RGC ribosomes from whole retina lysates directly without the need of retinal cell dissociation and RGC purification. Deep sequencing of translating mRNAs isolated from RGC ribosomes showed high in group correlation and dramatic differences between naïve and glaucomatous RGCs one week post SO injection (1 wpi) (FIG. 1B). The GO analysis of the differentially expressed genes (DEGs) identified multiple cellular pathways that have been significantly changed in the glaucomatous RGCs (FIG. 1C); we focused on genes involved in NAD+ metabolism (FIG. 1D,E): All three isoforms of NMNATs can be detected in RGCs but NMNAT2 is the most abundant; NMNAT2, but not NMNAT1 or NMNAT3, is significantly decreased in glaucomatous RGCs, suggesting the primary role of NMNAT2 downregulation in glaucomatous neurodegeneration. NAPRT (nicotinic acid phosphoribosyltransferase) is the enzyme that generates the substrate of NMNAT by converting nicotinic acid to NAMN. NAPRT is upregulated in glaucomatous RGCs (FIG. 1E), which may be a compensatory response to the decrease in NMNAT2. In summary, NMNAT2 is the major isoform of NMNAT in RGCs and its decline is an early event occurring shortly after the onset of ocular hypertension and before significant neurodegeneration.

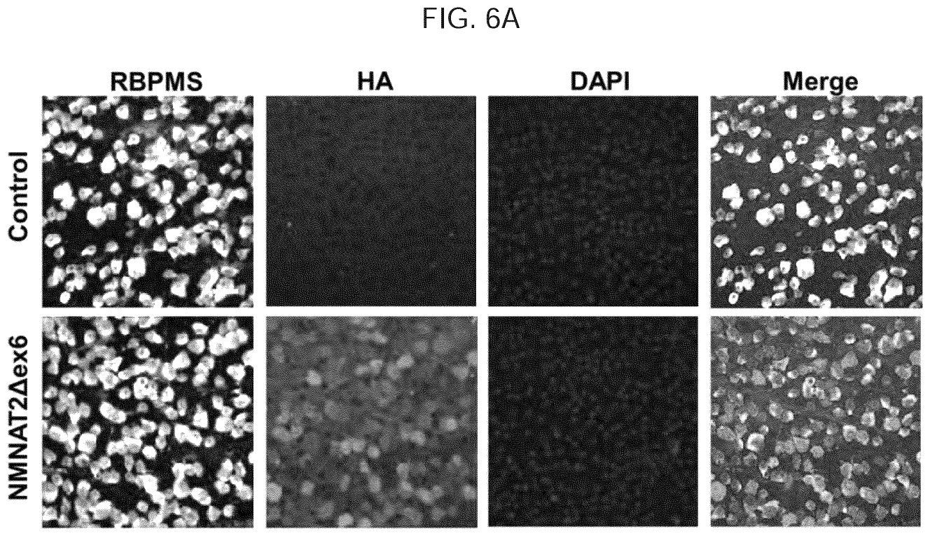

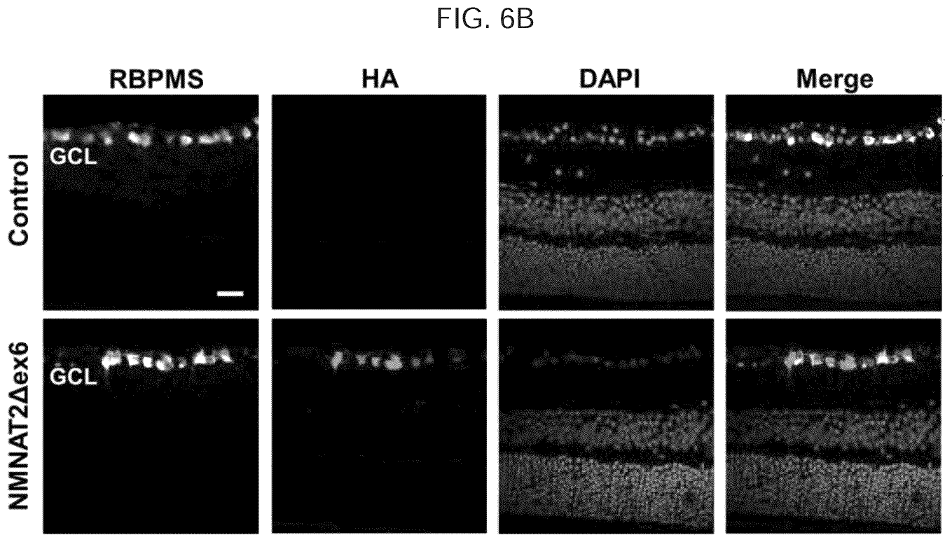

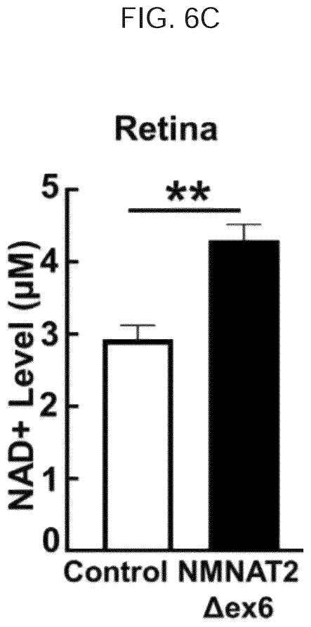

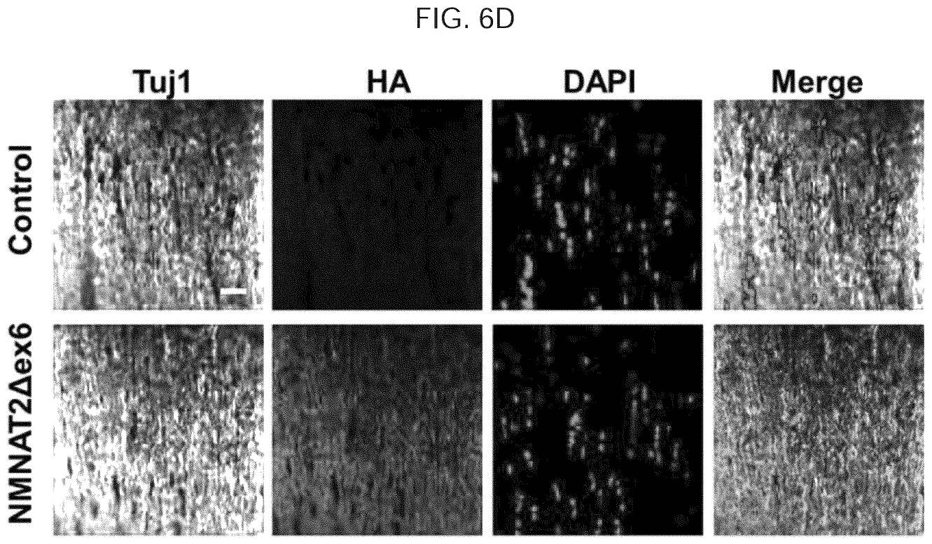

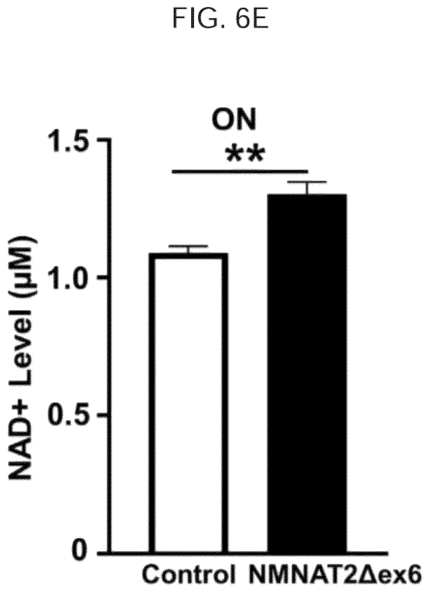

RGC-specific expression of NMNAT2Δex6 and increase of NAD+ in both retina and ON. Because NMNAT2 protein is very labile and rapidly depleted after axotomy, downregulation of axonal NAD+ is known to cause axon degeneration. Two E3 ubiquitin ligases, PHR1/Highwire and SCF, are involved in NMNAT2 degradation and axonal degeneration. Our finding that the mRNA level of NMNAT2 in RGCs is decreased by ocular hypertension further supports the notion that the lack of NMNAT2 contributes to glaucomatous neurodegeneration. Therefore, we next examined the effect of RGC-specific NMNAT2 overexpression on RGCs' survival after injury and disease. NMNAT2Δex6 (soluble forms of NMNAT2, lacking of exon 6) is more stable and has greater axon protective capacity than wild type NMNAT2. We engineered an AAV vector to drive NMNAT2Δex6 under the mSncg promoter and confirmed RGC-specific expression of NMNAT2 after intravitreal injection: HA-tagged NMNAT2 colocalized with RBPMS+ RGCs and Tuj1+ ONs, but not with other layers of retina (FIG. 6A,B,D). We further confirmed that NMNAT2 overexpression increased NAD+ levels significantly in both retina and ON (FIG. 6C,E). In summary, we established RGC-specific upregulation of NMNAT2, which will enable us to evaluate the RGC autonomous effect of NMNAT2 modulation in glaucomatous neuroprotection.

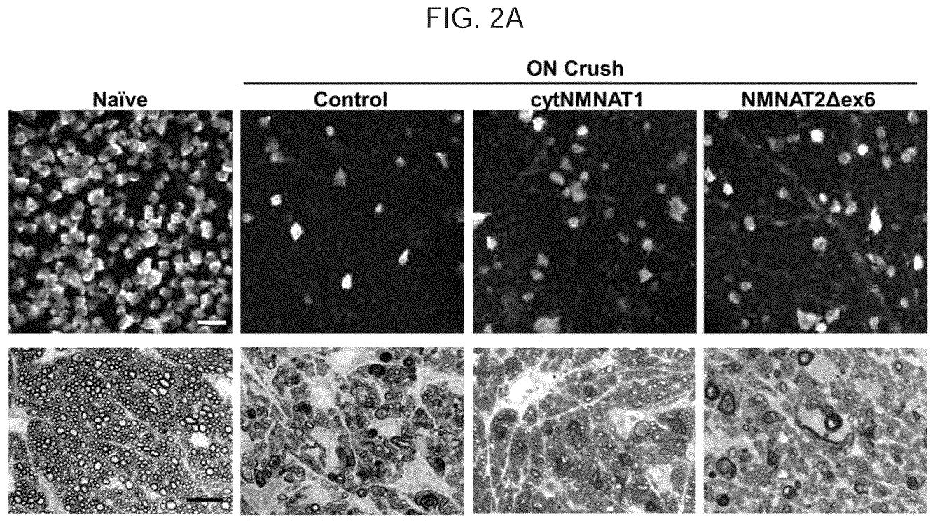

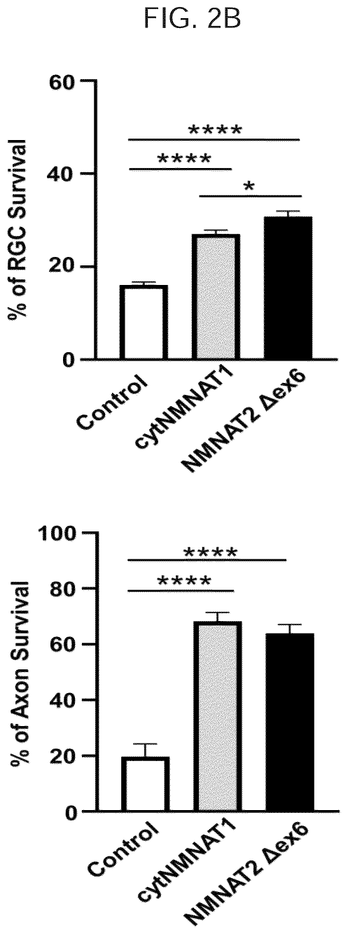

NMNAT2 overexpression significantly promotes both RGC soma and axon survival after ON crush injury. ON crush is extensively used as a convenient optic neuropathy model that injures all RGC axons and causes universal RGC degeneration; it also often serves as an acceptable surrogate glaucoma model. To determine whether a neuronal autonomous effect of NMNAT2 provides RGC and ON protection, we first injected AAV2-NMNAT2 or AAV2 control into the vitreous of the left eyes of naïve mice two weeks before ON crush. Compared to the contralateral naïve control eyes, ON crush causes significant loss of RGC somata and axons (FIG. 2A). Consistent with the previous report that the pan-neuronal overexpression of cytNMNAT1 in cytosol and axon protects both RGC somata and axons in optic neuropathy models, we found that cytNMNAT1 overexpression also promotes RGC soma and axon survival after a traumatic injury, ON crush (FIG. 2A,B). Interestingly, NMNAT2 overexpression protects RGC soma significantly better than NMNAT1, but there is no significant difference in RGC axon protection (FIG. 2A,B).

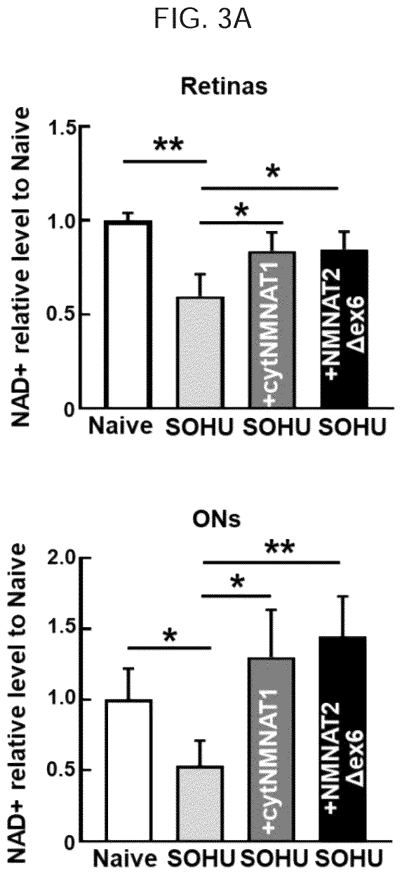

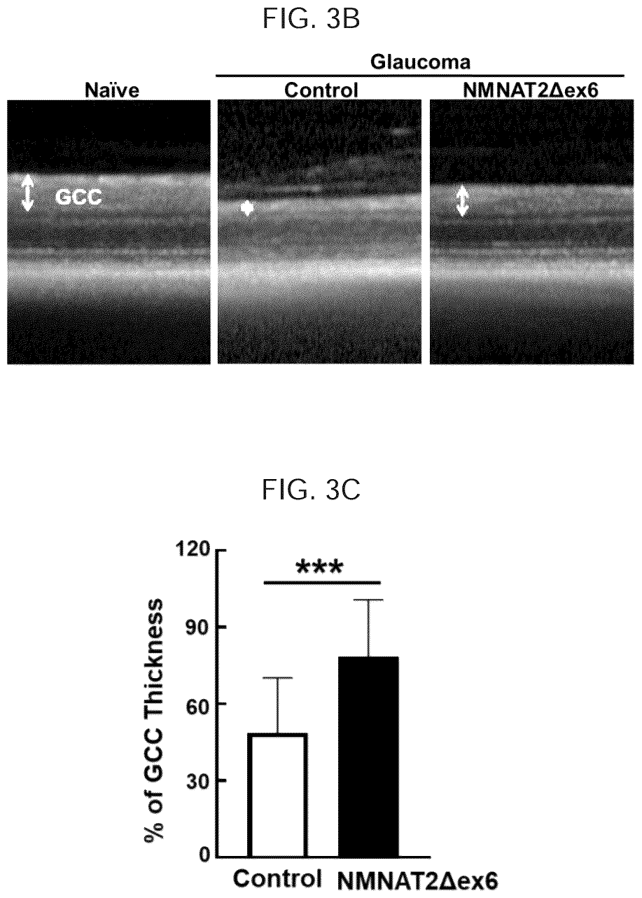

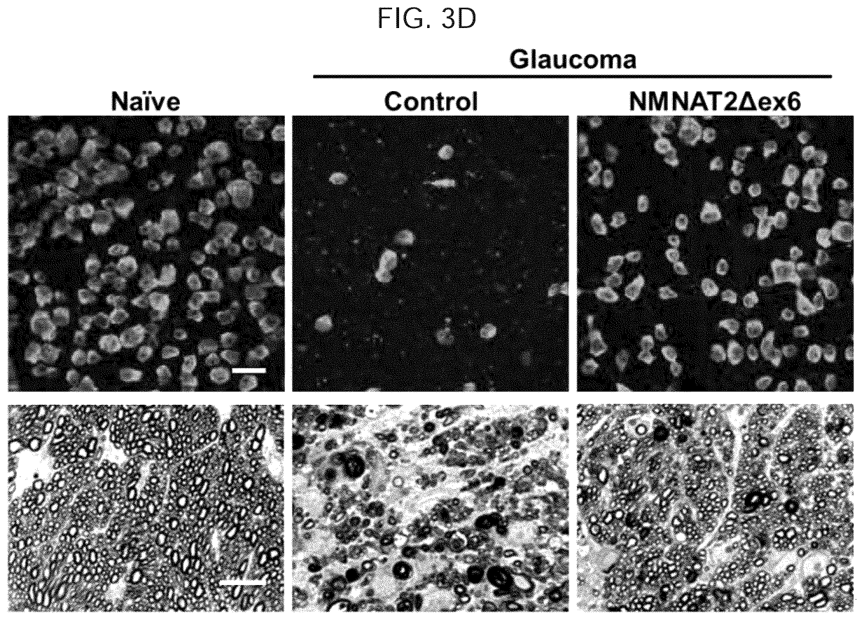

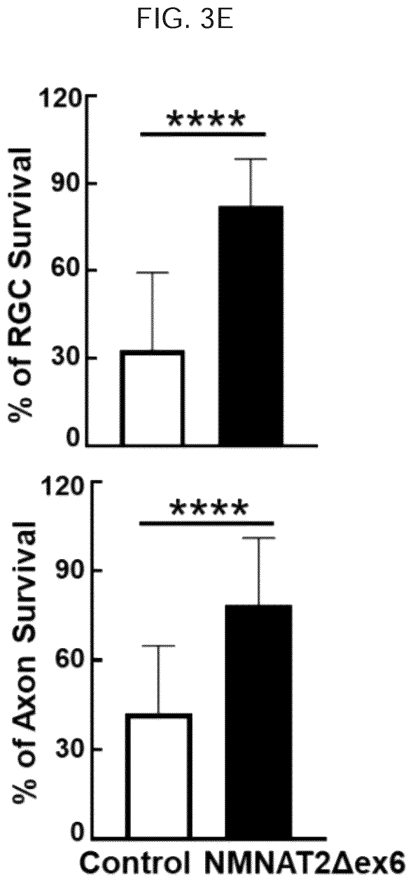

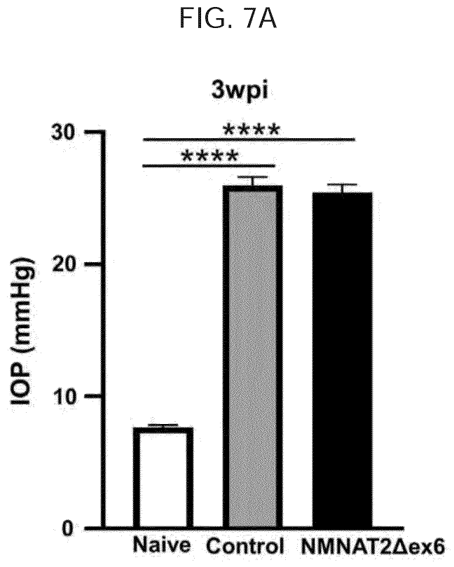

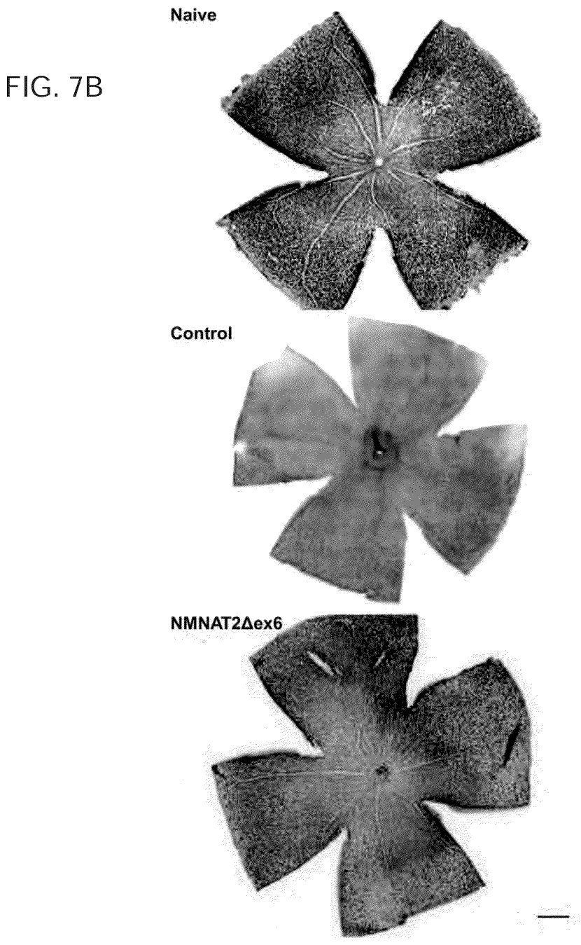

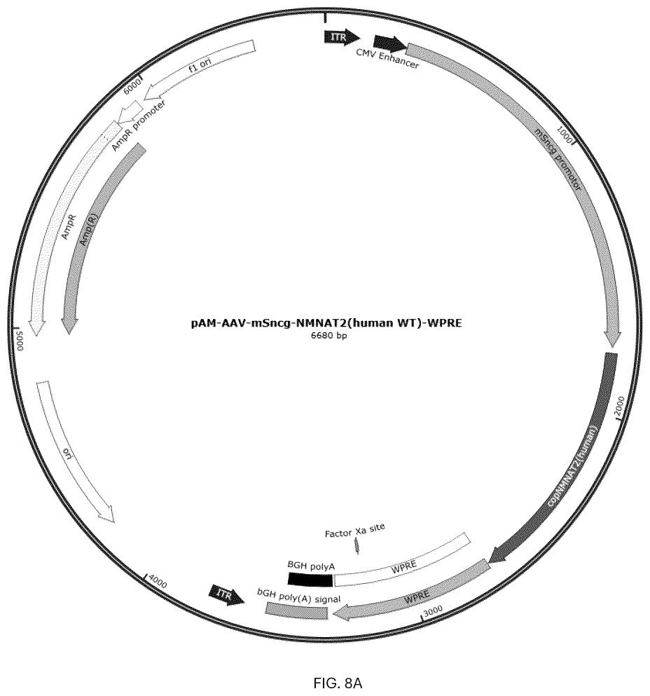

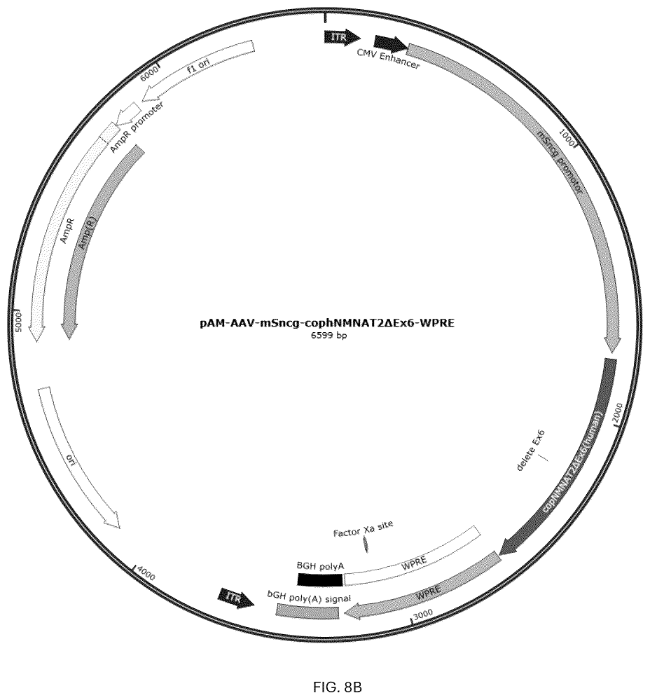

NMNAT2 overexpression significantly promotes both RGC soma and axon survival in glaucoma. To determine whether neuronal autonomous NMNAT2 protects in glaucoma, we first performed intravitreal injection of AAV2-NMNAT2 or AAV2 control in the left eyes of naïve mice three weeks before SO injection. OCT showed significant thinning of the ganglion cell complex (GCC), including both RGC dendrites and axons, in living animals 3 weeks post SO injection (3 wpi) (FIG. 3A,B), concurrent with significant IOP elevation (FIG. 7A). Histological analysis of post-mortem retina wholemounts and ON sections consistently demonstrated significant loss of RGC somata (FIG. 3C,D and FIG. 7B). RGC-specific expression of NMNAT2 promotes dramatic survival of both RGC somata and axons (FIG. 3A-D and FIG. 7B). Taken together, NMNAT2 overexpression in RGCs achieves significant neuroprotection of RGCs and ONs in both traumatic ON injury and glaucoma.

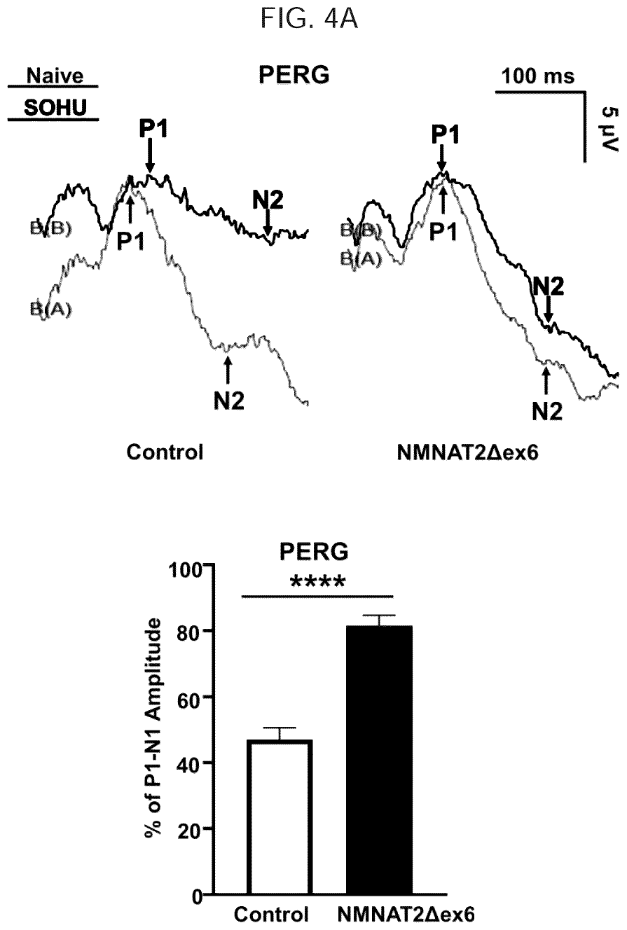

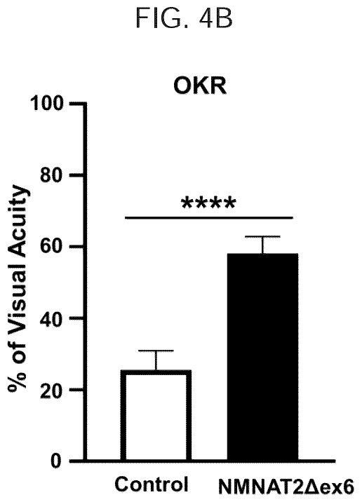

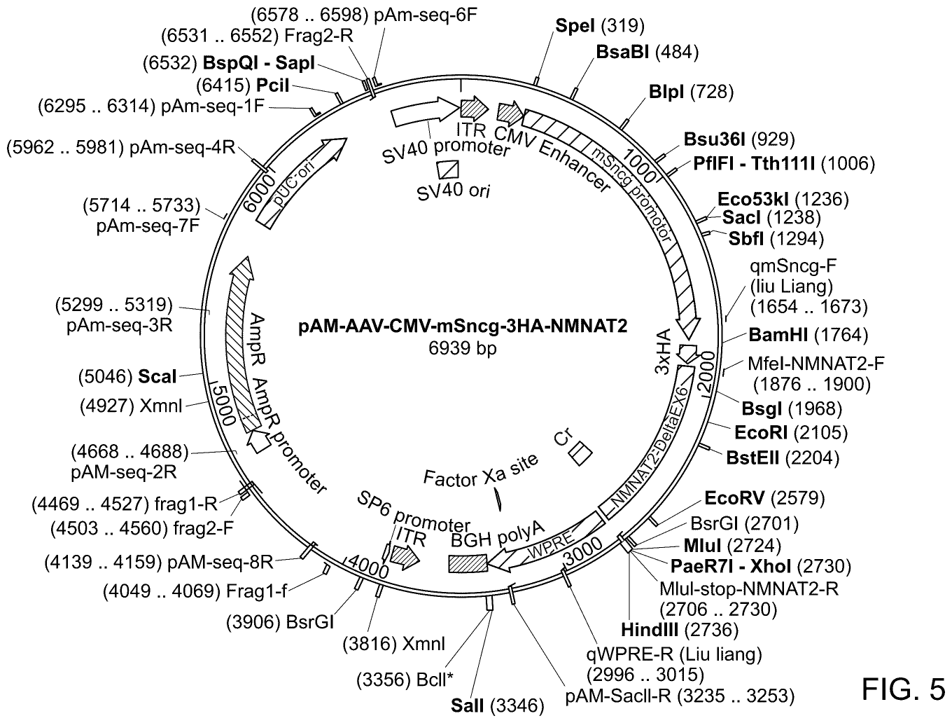

NMNAT2 overexpression preserves visual functions of glaucomatous mice. In addition to morphological protection, we also investigated whether RGC-specific expression of NMNAT2 preserved visual function in glaucomatous mice. We used PERG to examine RGC function and OKR for visual acuity: both techniques are well established in our lab. Consistent with our morphological and histological results, NMNAT2 significantly preserved visual function in glaucomatous eyes, improving the amplitude of PERG (FIG. 4A) and visual acuity compared to controls (FIG. 4B).

In this study, we determined the expression levels of the genes involved in NAD+ metabolism in both naïve and glaucomatous RGCs. NMNAT2 is the dominant form of NMNATs in RGCs and its mRNA level, but not that of NMNAT1 or NMNAT3, is significant decreased in glaucomatous RGCs. We demonstrated that intravitreal injection of AAV2-mSncg-NMNAT2Δex6 increases NMNAT2 expression and NAD+ levels specifically in RGCs and ONs. We then tested this gene therapy strategy in two optic neuropathy models, traumatic ON crush and ocular hypertension glaucoma. RGC-specific NMNAT2 overexpression significantly promotes survival of RGC somata and axons in both models and preserves visual function in glaucoma. These results contrast dramatically with that of another study showing that NMNAT2 overexpression fails to provide neuroprotection in an EAE/optic neuritis model, suggesting that NMNAT2 dysfunction may be uniquely associated with traumatic and glaucomatous retina/ON injuries.

The axonal NAD+level declines rapidly in injured axons, primarily due to depletion of the axonal NAD+-synthetic enzyme NMNAT2 and activation of the NAD+-consuming enzyme SARM1 (sterile alpha and TIR motif-containing protein 1), a downstream acting pro-degeneration factor for NMNAT2. Both the short half-life of NMNAT2 protein identified before and decreased NMNAT2 transcription in glaucoma identified by this study (FIG. 1E) may contribute to the NMNAT2 depletion and therefore cause axon degeneration. An additional factor that may also contribute is the decreased axonal transport of NMNAT2 found with aging, a common risk factor in chronic neurodegenerative diseases. Significantly reduced levels of NMNAT2 mRNA and protein have been identified consistently in Alzheimer disease, and loss of function NMNAT2 mutations have been detected in rare neurological diseases.

A causative NMNAT2 mutation has not been found yet for glaucoma, however, germline deletion of NMNAT2 causes ON truncation in mice and multiple NMNAT1 mutations cause Leber congenital amaurosis type 9 (LCA9), an autosomal recessive photoreceptor degenerative disease. The mutations in LCA9 probably act through photoreceptor-autonomous effects because pan-neuronal NMNAT1 deletion specifically causes degeneration of photoreceptors, but not of RGCs. The observations that NMNAT2 is the predominant NMNAT isoform in RGCs and that it provides better RGC protection than NMNAT1 (FIG. 3). NMNAT2 provides an excellent therapeutic target for RGC neuroprotection.

Taken together, our evidence shows that a low NMNAT2 level is pro-neurodegenerative and that strategies boosting neuronal intrinsic NMNAT2 levels provide a therapeutic treatment for glaucoma and other axonopathies. The retina is readily accessible for administration of a virus, which is applied by a localized injection that is confined to the eye and requires minimal virus load and causes minimal, if any, systemic effects. Considered along with these other advantages, our demonstration that AAV-mediated RGC-specific promoter mSncg-driven NMNAT2 overexpression provides significant neuroprotection of glaucomatous RGCs/ONs and preservation of visual functions establishes a translatable gene therapy approach to glaucoma.

SARM1 is a Toll-like receptor adapter protein but with intrinsic NAD₊ hydrolase activity that causes axon degeneration by degrading axonal NAD₊ significantly after injury-induced activation. Importantly, SARM1 deletion rescues NMNAT2-deficient axons, indicating that SARM1 works downstream of NMNAT2 depletion. Although we did not detect significant increase of SARM1 transcription in glaucomatous RGCs, blocking SARM1 activity is another promising strategy for neuroprotection, as demonstrated in many but not all neurodegenerative disease models. Germline deletion of SARM1 in mouse delays ON degeneration significantly after traumatic crush injury, but has no effect on RGC survival. We are testing whether RGC-specific SARM1 inhibition protects against glaucomatous neurodegeneration.

In summary, we found decreased NMNAT2 expression in glaucomatous RGCs, which can lead to NAD+ decline in RGCs and ONs. We further demonstrated that modulating RGC intrinsic levels of NMNAT2 by an AAV2-mSncg vector provides a potent gene therapy strategy for protecting both RGC somata and axons in traumatic ON injury and glaucoma.

### Materials and Methods

Animals. C57BL/6J WT (#000664) and RiboTag (011029) mice (7-9 weeks old) were purchased from Jackson Laboratories (Bar Harbor, Maine) and housed in standard cages on a 12 hours light-dark cycle. All experimental procedures were performed in compliance with animal protocols approved by the IACUC at Stanford University School of Medicine.

Constructs. The AAV2 vector containing the mSncg promoter, NMNAT2Δex6 and cytNMNAT1 has been described before. The 3HA-NMNAT2Δex6 is driven by the mSncg promoter and the cytNMNAT1 is driven by the universal CAG promoter.

AAV production and intravitreal injection. The detailed procedure of AAV production has been described previously. The AAV titers were determined by real-time PCR and diluted to 1.5×10¹² vector genome (vg)/ml. For intravitreal injection, mice were anesthetized by xylazine and ketamine based on their body weight (0.01 mg xylazine/g+0.08 mg ketamine/g). A pulled and polished microcapillary needle was inserted into the peripheral retina just behind the ora serrata. Approximately 2 μl of the vitreous was removed to allow injection of 2 μl AAV into the vitreous chamber to achieve 3×10⁹ vg/retina.

ON crush model. ON crush was performed 2 weeks following AAV injection: the ON was exposed intraorbitally while care was taken not to damage the underlying ophthalmic artery, and crushed with a jeweler's forceps (Dumont #5; Fine Science Tools, Foster City, California) for 5 seconds approximately 0.5 mm behind the eyeball. Eye ointment containing neomycin (Akorn, Somerset, New Jersey) was applied to protect the cornea after surgery.

SOHU glaucoma model and IOP measurement: Silicone oil-induced ocular hypertension (SOHU) mouse models and IOP measurement have been detailed before. In brief, mice were anesthetized by an intraperitoneal injection of Avertin (0.3 mg/g) and received the SO (Alcon Laboratories, 1,000 mPa·s) injection at 9-10 weeks of age. Prior to injection, one drop of 0.5% proparacaine hydrochloride (Akorn, Somerset, New Jersey) was applied to the cornea to reduce its sensitivity during the procedure. A 32 G needle was tunneled through the layers of the cornea at the superotemporal side close to the limbus to reach the anterior chamber without injuring lens or iris. Following this entry, ˜2 μl silicone oil (1,000 mPa·s, Silikon, Alcon Laboratories, Fort Worth, Texas) was injected slowly into the anterior chamber using a homemade sterile glass micropipette, until the oil droplet expanded to cover most areas of the iris (diameter˜1.8-2.2 mm). After the injection, veterinary antibiotic ointment (BNP ophthalmic ointment, Vetropolycin, Dechra, Overland Park, Kansas) was applied to the surface of the injected eye. The contralateral control eyes received mock injection with 2 μl normal saline to the anterior chamber. Throughout the procedure, artificial tears (Systane Ultra Lubricant Eye Drops, Alcon Laboratories, Fort Worth, Texas) were applied to keep the cornea moist.

The detailed procedure for IOP measurement has been described before. The IOP of both eyes was measured by the TonoLab tonometer (Colonial Medical Supply, Espoo, Finland) according to product instructions under a sustained flow of isoflurane (3% isoflurane at 2 L/minute mixed with oxygen) delivered to the nose by a special rodent nose cone (Xenotec, Inc., Rolla, Missouri). 1% Tropicamide Sterile Ophthalmic Solution (Akorn, Somerset, New Jersey) was applied three times at 3-minute intervals to fully dilate the pupils (about 10 minutes) before taking measurements. During this procedure, artificial tears were applied to keep the cornea moist. Since IOP measurement requires pupil dilation, which essentially relieves the ocular hypertension during the period of pupil dilation, we only measure IOP 3 weeks after SO injection immediately before sacrificing the animals in the acute and severe ND (no dilation) SOHU model that we described before.

RiboTag immunoprecipitation (Ribo-IP), RNA extraction, RNA-seq and data analysis. RiboTag mice (Jackson Laboratory, B6N.129-Rpl22tm1.1Psam) were intravitreally injected with 2 μl AAV2-mSncg-Cre (1.5×10¹² vg/ml) to achieve RGC ribosome labeling. Three weeks after the AAV injection, SOHU model mice were prepared as previously described. Freshly isolated retinas (10-12 retinas/condition) at 1 wpi in SOHU eyes and contralateral control eyes were homogenized with a pestle in homogenization buffer (50 mM Tris HCl, 100 mM KCl, 12 mM MgCl₂, 1% NP-40, 1 mM DTT, 1× protease inhibitor (Sigma), 200 U/ml RNAsin (Promega, Madison, WI), and 100 μg/ml cycloheximide, 1 mg/ml heparin). Samples were then centrifuged at 12,000 g for 2 minutes and the supernatant used for IP. 10 μl anti-HA antibody (BioLegend, San Diego, CA) was added into each sample and incubated for 4 hours at room temperature with rotation before incubation with Protein G magnetic beads (prewashed with homogenization buffer, Thermo Fisher Scientific, South San Francisco, CA) overnight at 4° C. with rotation. Sample tubes were placed in a magnetic adaptor to aggregate the magnetic beads and the supernatant was discarded. The beads were washed for 10 minutes×3 in a high-salt buffer (50 mM Tris HCl, 300 mM KCl, 12 mM MgCl2, 1% NP-40, 1 mM DTT, 100 μg/ml cycloheximide) before resuspension in Trizol (Thermo Fisher Scientific, South San Francisco, CA) for RNA isolation with PureLink RNA Mini Kit (Thermo Fisher Scientific, South San Francisco, CA) following the manufacturer's protocol. RNA integrity was analyzed using a 2100 Bioanalyzer (Agilent, Santa Clara, CA) with the RNA Pico chip; the samples with RNA integrity number (RIN) greater than 6 were used for library preparation. Library preparation and sequencing were performed using the ultra-low input RNA-seq service from GENEWIZ (South Plainfield, NJ). Briefly, the cDNA was generated using the SMART-Seq v4 Ultra Low Input RNA kit (Takara, Mountain View, CA). Libraries were then constructed using the Illumina Nextera XT kit (Illumina, San Diego, CA) and sequencing performed on the Illumina HiSeq 4000 sequencer (Illumina, San Diego, CA) with 2×150 bp paired-end configuration. Three biological replicate samples were prepared and sequenced for each condition.

RNA-seq raw data were trimmed by trim-galore to remove adaptor sequences and aligned with Hisat2 to the mouse reference genome (version mm10). The aligned reads ranges are from 59.7 million (93.36%) to 67.1 million (94.25%). MultiQC was used to assess the quality of the sequence data. The count matrix of all the genes in the individual samples was determined by Feature Counts. Samples were further processed by DEseq2 package working in R environment to determine differentially expressed genes (DEGs) between SOHU RGCs vs naïve RGCs. 1132 DEGs in SOHU were detected with the cutoff, >1.2-fold change upregulated or <0.7-fold downregulated, and adjusted p value<0.05. A heatmap was generated using pheatmap package to illustrate the DEGs of different groups. We used clusterProfiler package working in R environment to perform enrichment analysis of gene clusters, and visualize the top enriched Gene Ontology (GO) items. The log₁₀ (adjust p-value) of the enriched biological process was plotted to indicate the significance of the enrichment of each item. All the raw data and processed data have been submitted to the Gene expression Omnibus (GEO). The accession number is GEO: GSE182483.

Immunohistochemistry of whole mount and cross sections of retina. The detailed procedures have been published before. Briefly, after perfusion fixation with 4% PFA in PBS, mice eyeballs and ONs were dissected out and post-fixed with 4% PFA for 2 hours at room temperature. 30% sucrose was then used for cryoprotection of the tissues. Retinas were dissected out for whole mount retina immunostaining. For cross sections of retina, the eyeballs were embedded in tissue-tek OCT on dry ice for subsequent cryo-section with a Leica cryostat. The primary antibodies used for immunostaining: anti-RBPMS at 1:4000 (Custom made at ProSci Inc); anti-HA at 1:200 (Roche, 11867423001); anti-Tuj1 at 1:200 (Biolegend,845502). Secondary antibodies were then applied (1:200; Jackson ImmunoResearch, West Grove, Pennsylvania) and incubated for 1 hour at room temperature before mounting.

RGC counting. The detailed procedures have been published before. For peripheral RGC counting, whole-mount retinas were immunostained with the RBPMS antibody, 8 fields sampled from peripheral regions of each retina using a 40× lens with a Zeiss M2 epifluorescence microscope, and RBPMS+ RGCs counted by Volocity software (Quorum Technologies). The percentage of RGC survival was calculated as the ratio of surviving RGC numbers in injured eyes compared to contralateral uninjured eyes. The investigators who counted the cells were masked to the treatment of the samples.

ON semi-thin sections and quantification of surviving axons. The detailed procedure of ON semi-thin section preparation and paraphenylenediamine (PPD) staining has been described previously. Briefly, ONs were post-fixed in situ with 2% glutaraldehyde and 2% PFA in 0.1 M PBS. Semi-thin (1 μm) cross sections of the ON 2 mm distal to the eye (globe) were collected. After PPD staining, four sections of each ON were imaged through a 100× lens of a Zeiss M2 epifluorescence microscope to cover the entire area of the ON without overlap. Two areas of 21.4 μm×29.1 μm were cropped from the center of each image, and the surviving axons within the designated areas counted manually. After counting all the images taken from a single nerve, the mean of the surviving axon number was calculated for each ON. The mean of the surviving axon number in the injured ON was compared to that in the contralateral control ON to yield a percentage of axon survival value. The investigators who counted the axons were masked to the treatment of the samples.

NAD+ measurement. The NAD+ levels of ONs were measured according to the manufacturer's protocol with the NAD+/NADH Assay Kit (Abnova, KA1657). Mice were sacrificed by cervical dislocation and retinas and ONs collected gently and quickly. One retina/sample or two ONs/sample were homogenized in NAD+ extraction buffer and then heated at 60° C. for 5 minutes. The homogenates added with an assay buffer were centrifuged at 14,000 rpm for 5 minutes to remove cellular debris. After adding reagents to 40 μl supernatants and standard solutions, the absorbance was determined at 565 nm by TECAN SPARK Plate Reader (Tecan, Switzerland). The results were normalized to a microgram of protein concentration.

Spectral-Domain Optical Coherence Tomography (SD-OCT) imaging. The detailed procedure has been published previously. Briefly, the retina fundus images were captured with the Heidelberg Spectralis SLO/OCT system (Heidelberg Engineering, Germany). The mouse retina was scanned with the ring scan mode centered by the optic nerve head under high-resolution mode (each B-scan consisted of 1536 A scans). The ganglion cell complex (GCC) includes retinal nerve fiber layer (RNFL), ganglion cell layer (GCL) and inner plexiform layer (IPL). The average thickness of GCC around the optic nerve head was measured manually with the aid of Heidelberg software. The investigators who measured the thickness of GCC were masked to the treatment of the samples.

Pattern Electroretinogram (PERG) recording. The detailed procedure has been published previously. Briefly, after anesthetization and pupil dilation, PERG of both eyes was recorded simultaneously with the Miami PERG system (Intelligent Hearing Systems, Miami, Florida) according to manufacturer's instructions. Two consecutive recordings of 200 traces were averaged to achieve one readout; each trace recorded up to 1020 ms. The first positive peak in the waveform was designated as P1 and the second negative peak as N2. The amplitude was measured from P1 to N2.

Optokinetic Tracking Response (OKR). The detailed procedure has been published previously. Briefly, mice were placed on a platform in the center of four 17-inch LCD computer monitors (Dell, Phoenix, AZ), with a video camera above the platform to capture the movement of the mouse. A rotating cylinder with vertical sine wave grating was computed and projected to the four monitors by OptoMotry software (CerebralMechanics Inc, Lethbridge, Alberta, Canada). The sine wave grating, settled at 100% contrast and speed of 12 degrees per second, provides a virtual-reality environment to measure the spatial acuity (cycle/degree) of the left eye when rotated clockwise and the right eye when rotated counterclockwise. The maximum frequency (cycle/degree) that the mouse could track was identified and recorded by investigators masked to treatment.

Statistical Analysis. GraphPad Prism 7 was used to generate graphs and for statistical analyses. Data are presented as means±s.e.m. Student's t-test was used for two groups comparison and One-way ANOVA with post hoc test was used for multiple comparisons.

### REFERENCES

Tham, Y. C., Li, X., Wong, T. Y., Quigley, H. A., Aung, T., and Cheng, C. Y. (2014). Global prevalence of glaucoma and projections of glaucoma burden through 2040: a systematic review and meta-analysis. Ophthalmology 121, 2081-2090.
Quigley, H. A., Nickells, R. W., Kerrigan, L. A., Pease, M. E., Thibault, D. J., and Zack, D.J. (1995). Retinal ganglion cell death in experimental glaucoma and after axotomy occurs by apoptosis. Invest Ophthalmol Vis Sci 36, 774-786.
Libby, R. T., Li, Y., Savinova, O. V., Barter, J., Smith, R. S., Nickells, R. W., and John, S. W. (2005). Susceptibility to neurodegeneration in a glaucoma is modified by Bax gene dosage. PLOS Genet 1, 17-26.
Howell, G. R., Libby, R. T., Jakobs, T. C., Smith, R. S., Phalan, F. C., Barter, J. W., Barbay, J. M., Marchant, J. K., Mahesh, N., Porciatti, V., Whitmore, A. V., et al. (2007). Axons of retinal ganglion cells are insulted in the optic nerve early in DBA/2J glaucoma. J Cell Biol 179, 1523-1537.
Nickells, R. W., Howell, G. R., Soto, I., and John, S. W. (2012). Under pressure: cellular and molecular responses during glaucoma, a common neurodegeneration with axonopathy. Annu Rev Neurosci 35, 153-179.
Jonas, J. B., Aung, T., Bourne, R. R., Bron, A. M., Ritch, R., and Panda-Jonas, S. (2017). Glaucoma. Lancet 390, 2183-2193. 10.1016/S0140-6736(17)31469-1.
Weinreb, R. N., Leung, C. K., Crowston, J. G., Medeiros, F. A., Friedman, D. S., Wiggs, J. L., and Martin, K. R. (2016). Primary open-angle glaucoma. Nat Rev Dis Primers 2, 16067.
Calkins, D. J. (2021). Adaptive responses to neurodegenerative stress in glaucoma. Prog Retin Eye Res, 100953. 10.1016/j.preteyeres.2021.100953.
Iyer, J., Vianna, J. R., Chauhan, B. C., and Quigley, H. A. (2020). Toward a new definition of glaucomatous optic neuropathy for clinical research. Curr Opin Ophthalmol 31, 85-90.
Mafwiri, M., Bowman, R. J., Wood, M., and Kabiru, J. (2005). Primary open-angle glaucoma presentation at a tertiary unit in Africa: intraocular pressure levels and visual status. Ophthalmic epidemiology 12, 299-302.
Burgoyne, C. F. (2011). A biomechanical paradigm for axonal insult within the optic nerve head in aging and glaucoma. Experimental eye research 93, 120-132.
Stowell, C., Burgoyne, C. F., Tamm, E. R., Ethier, C. R., Lasker, I. I. o. A., and Glaucomatous Neurodegeneration, P. (2017). Biomechanical aspects of axonal damage in glaucoma: A brief review. Experimental eye research 157, 13-19.
Heijl, A., Leske, M. C., Bengtsson, B., Hyman, L., Bengtsson, B., Hussein, M., and Early 488 Manifest Glaucoma Trial, G. (2002). Reduction of intraocular pressure and glaucoma progression: results from the Early Manifest Glaucoma Trial. Arch Ophthalmol 120, 1268-490 1279.
Garway-Heath, D. F., Crabb, D. P., Bunce, C., Lascaratos, G., Amalfitano, F., Anand, N., Azuara-Blanco, A., Bourne, R. R., Broadway, D. C., Cunliffe, I. A., Diamond, J. P., et al. (2015). Latanoprost for open-angle glaucoma (UKGTS): a randomised, multicentre, placebo-controlled trial. Lancet 385, 1295-1304.
Anderson, D. R., and Normal Tension Glaucoma, S. (2003). Collaborative normal tension glaucoma study. Curr Opin Ophthalmol 14, 86-90.
Wormald, R., Virgili, G., and Azuara-Blanco, A. (2020). Systematic reviews and randomised controlled trials on open angle glaucoma. Eye (Lond) 34, 161-167.
Howell, G. R., Soto, I., Libby, R. T., and John, S. W. (2013). Intrinsic axonal degeneration pathways are critical for glaucomatous damage. Exp Neurol 246, 54-61.
Figley, M. D., and DiAntonio, A. (2020). The SARM1 axon degeneration pathway: control of the NAD₍₊₎ metabolome regulates axon survival in health and disease. Curr Opin Neurobiol 505 63, 59-66.
Sambashivan, S., and Freeman, M. R. (2021). SARM1 signaling mechanisms in the injured nervous system. Curr Opin Neurobiol 69, 247-255.
Coleman, M. P., and Hoke, A. (2020). Programmed axon degeneration: from mouse to mechanism to medicine. Nat Rev Neurosci 21, 183-196.
Wang, J., Zhai, Q., Chen, Y., Lin, E., Gu, W., McBurney, M. W., and He, Z. (2005). A local mechanism mediates NAD-dependent protection of axon degeneration. J Cell Biol 170, 349-355.
Wang, J. T., Medress, Z. A., Vargas, M. E., and Barres, B. A. (2015). Local axonal protection by WldS as revealed by conditional regulation of protein stability. Proc Natl Acad Sci USA 112, 10093-10100.
Gerdts, J., Brace, E. J., Sasaki, Y., DiAntonio, A., and Milbrandt, J. (2015). SARM1 activation triggers axon degeneration locally via NAD(+) destruction. Science 348, 453-457.
Mack, T. G., Reiner, M., Beirowski, B., Mi, W., Emanuelli, M., Wagner, D., Thomson, D., Gillingwater, T., Court, F., Conforti, L., Fernando, F. S., et al. (2001). Wallerian degeneration of injured axons and synapses is delayed by a Ube4b/Nmnat chimeric gene. Nat Neurosci 4, 522 1199-1206.
Sasaki, Y., Vohra, B. P., Baloh, R. H., and Milbrandt, J. (2009). Transgenic mice expressing the Nmnat1 protein manifest robust delay in axonal degeneration in vivo. J Neurosci 29, 6526-6534.
Beirowski, B., Babetto, E., Coleman, M. P., and Martin, K. R. (2008). The WldS gene delays axonal but not somatic degeneration in a rat glaucoma model. The European journal of neuroscience 28, 1166-1179.
Williams, P. A., Harder, J. M., Foxworth, N. E., Cardozo, B. H., Cochran, K. E., and John, S. W. M. (2017). Nicotinamide and WLD(S) Act Together to Prevent Neurodegeneration in Glaucoma. 531 Front Neurosci 11, 232.
Risner, M. L., Pasini, S., McGrady, N. R., D'Alessandro, K. B., Yao, V., Cooper, M. L., and Calkins, D. J. (2021). Neuroprotection by Wld(S) depends on retinal ganglion cell type and age in glaucoma. Mol Neurodegener 16, 36.
Williams, P. A., Harder, J. M., Foxworth, N. E., Cochran, K. E., Philip, V. M., Porciatti, V., Smithies, O., and John, S. W. (2017). Vitamin B3 modulates mitochondrial vulnerability and prevents glaucoma in aged mice. Science 355, 756-760.
Zhu, Y., Zhang, L., Sasaki, Y., Milbrandt, J., and Gidday, J. M. (2013). Protection of mouse retinal ganglion cell axons and soma from glaucomatous and ischemic injury by cytoplasmic overexpression of Nmnat1. Invest Ophthalmol Vis Sci 54, 25-36.
Berger, F., Lau, C., Dahlmann, M., and Ziegler, M. (2005). Subcellular compartmentation and differential catalytic properties of the three human nicotinamide mononucleotide adenylyltransferase isoforms. J Biol Chem 280, 36334-36341.
Cahoy, J. D., Emery, B., Kaushal, A., Foo, L. C., Zamanian, J. L., Christopherson, K. S., Xing, Y., Lubischer, J. L., Krieg, P. A., Krupenko, S. A., Thompson, W. J., et al. (2008). A 546 transcriptome database for astrocytes, neurons, and oligodendrocytes: a new resource for 547 understanding brain development and function. J Neurosci 28, 264-278.
Gilley, J., and Coleman, M. P. (2010). Endogenous Nmnat2 is an essential survival factor for maintenance of healthy axons. PLOS biology 8, e1000300.
Gilley, J., Adalbert, R., Yu, G., and Coleman, M. P. (2013). Rescue of peripheral and CNS axon defects in mice lacking NMNAT2. J Neurosci 33, 13410-13424.
Feng, Y., Yan, T., Zheng, J., Ge, X., Mu, Y., Zhang, Y., Wu, D., Du, J. L., and Zhai, Q. (2010). Overexpression of Wld(S) or Nmnat2 in mauthner cells by single-cell electroporation delays axon degeneration in live zebrafish. Journal of neuroscience research 88, 3319-3327.
Yan, T., Feng, Y., Zheng, J., Ge, X., Zhang, Y., Wu, D., Zhao, J., and Zhai, Q. (2010). Nmnat2 delays axon degeneration in superior cervical ganglia dependent on its NAD synthesis activity. Neurochem Int 56, 101-106.
Ali, Y. O., Allen, H. M., Yu, L., Li-Kroeger, D., Bakhshizadehmahmoudi, D., Hatcher, A., McCabe, C., Xu, J., Bjorklund, N., Taglialatela, G., Bennett, D. A., et al. (2016). NMNAT2:HSP90 Complex Mediates Proteostasis in Proteinopathies. PLOS biology 14, e1002472.
Zhang, J., Li, L., Huang, H., Fang, F., Webber, H. C., Zhuang, P., Liu, L., Dalal, R., Tang, P. H., Mahajan, V. B., Sun, Y., et al. (2019). Silicone oil-induced ocular hypertension and glaucomatous neurodegeneration in mouse. eLife 8.
Zhang, J., Fang, F., Li, L., Huang, H., Webber, H. C., Sun, Y., Mahajan, V. B., Hu, Y. (2019). A Reversible Silicon Oil-Induced Ocular Hypertension Model in Mice. J. Vis. Exp. 153.
Fang, F., Zhang, J., Zhuang, P., Liu, P., Li, L., Huang, H., Webber, H. C., Xu, Y., Liu, L., Dalal, R., Sun, Y., et al. (2021). Chronic mild and acute severe glaucomatous neurodegeneration derived from silicone oil-induced ocular hypertension. Scientific reports 11, 9052.
Wang, Q., Zhuang, P., Huang, H., Li, L., Liu, L., Webber, H. C., Dalal, R., Siew, L., Fligor, C. M., Chang, K. C., Nahmou, M., et al. (2020). Mouse gamma-Synuclein Promoter-Mediated Gene Expression and Editing in Mammalian Retinal Ganglion Cells. J Neurosci 40, 3896-3914.
Sanz, E., Yang, L., Su, T., Morris, D. R., McKnight, G. S., and Amieux, P. S. (2009). Cell-type-specific isolation of ribosome-associated mRNA from complex tissues. Proc Natl Acad Sci USA 106, 13939-13944.
Verdin, E. (2015). NAD₍₊₎ in aging, metabolism, and neurodegeneration. Science 350, 1208-1213.
Milde, S., Gilley, J., and Coleman, M. P. (2013). Subcellular localization determines the stability and axon protective capacity of axon survival factor Nmnat2. PLOS biology 11, e1001539.
Babetto, E., Beirowski, B., Russler, E. V., Milbrandt, J., and DiAntonio, A. (2013). The Phr1 ubiquitin ligase promotes injury-induced axon self-destruction. Cell reports 3, 1422-1429.
Xiong, X., Hao, Y., Sun, K., Li, J., Li, X., Mishra, B., Soppina, P., Wu, C., Hume, R. I., and Collins, C. A. (2012). The Highwire ubiquitin ligase promotes axonal degeneration by tuning levels of Nmnat protein. PLOS biology 10, e1001440.
Yamagishi, Y., and Tessier-Lavigne, M. (2016). An Atypical SCF-like Ubiquitin Ligase Complex Promotes Wallerian Degeneration through Regulation of Axonal Nmnat2. Cell reports 17, 774-782.
Milde, S., Fox, A. N., Freeman, M. R., and Coleman, M. P. (2013). Deletions within its subcellular targeting domain enhance the axon protective capacity of Nmnat2 in vivo. Scientific reports 3, 2567.
Li, L., Huang, H., Fang, F., Liu, L., Sun, Y., and Hu, Y. (2020). Longitudinal Morphological and Functional Assessment of RGC Neurodegeneration After Optic Nerve Crush in Mouse. Frontiers in cellular neuroscience 14, 109.
Milde, S., Adalbert, R., Elaman, M. H., and Coleman, M. P. (2015). Axonal transport declines with age in two distinct phases separated by a period of relative stability. Neurobiology of aging 36, 971-981.
Huppke, P., Wegener, E., Gilley, J., Angeletti, C., Kurth, I., Drenth, J. P. H., Stadelmann, C., Barrantes-Freer, A., Bruck, W., Thiele, H., Nurnberg, P., et al. (2019). Homozygous NMNAT2 mutation in sisters with polyneuropathy and erythromelalgia. Exp Neurol 320, 112958.
Lukacs, M., Gilley, J., Zhu, Y., Orsomando, G., Angeletti, C., Liu, J., Yang, X., Park, J., Hopkin, R. J., Coleman, M. P., Zhai, R. G., et al. (2019). Severe biallelic loss-of-function mutations in nicotinamide mononucleotide adenylyltransferase 2 (NMNAT2) in two fetuses with fetal akinesia deformation sequence. Exp Neurol 320, 112961.
Gilley, J., Orsomando, G., Nascimento-Ferreira, I., and Coleman, M. P. (2015). Absence of SARM1 rescues development and survival of NMNAT2-deficient axons. Cell reports 10, 1974-615 1981.
Falk, M. J., Zhang, Q., Nakamaru-Ogiso, E., Kannabiran, C., Fonseca-Kelly, Z., Chakarova, C., 617 Audo, I., Mackay, D. S., Zeitz, C., Borman, A. D., Staniszewska, M., et al. (2012). NMNAT1 mutations cause Leber congenital amaurosis. Nature genetics 44, 1040-1045.
Perrault, I., Hanein, S., Zanlonghi, X., Serre, V., Nicouleau, M., Defoort-Delhemmes, S., Delphin, N., Fares-Taie, L., Gerber, S., Xerri, O., Edelson, C., et al. (2012). Mutations in NMNAT1 cause Leber congenital amaurosis with early-onset severe macular and optic atrophy. Nature genetics 44, 975-977.
Koenekoop, R. K., Wang, H., Majewski, J., Wang, X., Lopez, I., Ren, H., Chen, Y., Li, Y., Fishman, G. A., Genead, M., Schwartzentruber, J., et al. (2012). Mutations in NMNAT1 cause Leber congenital amaurosis and identify a new disease pathway for retinal degeneration. Nature genetics 44, 1035-1039.
Chiang, P. W., Wang, J., Chen, Y., Fu, Q., Zhong, J., Chen, Y., Yi, X., Wu, R., Gan, H., Shi, Y., Chen, Y., et al. (2012). Exome sequencing identifies NMNAT1 mutations as a cause of Leber congenital amaurosis. Nature genetics 44, 972-974.
Kaplan, J., Perrault, I., Hanein, S., Dollfus, H., and Rozet, J. M. (2013). [Mutations in NMNAT1 cause Leber congenital amaurosis with severe macular and optic atrophy]. Med Sci (Paris) 29, 26-27.
Sasaki, Y., Kakita, H., Kubota, S., Sene, A., Lee, T. J., Ban, N., Dong, Z., Lin, J. B., Boye, S. L., DiAntonio, A., Boye, S. E., et al. (2020). SARM1 depletion rescues NMNAT1-dependent photoreceptor cell death and retinal degeneration. eLife 9.
Eblimit, A., Zaneveld, S. A., Liu, W., Thomas, K., Wang, K., Li, Y., Mardon, G., and Chen, R. (2018). NMNAT1 E257K variant, associated with Leber Congenital Amaurosis (LCA9), causes a mild retinal degeneration phenotype. Experimental eye research 173, 32-43.
Essuman, K., Summers, D. W., Sasaki, Y., Mao, X., DiAntonio, A., and Milbrandt, J. (2017). The SARM1 Toll/Interleukin-1 Receptor Domain Possesses Intrinsic NAD(+) Cleavage Activity that Promotes Pathological Axonal Degeneration. Neuron 93, 1334-1343 e1335.
Osterloh, J. M., Yang, J., Rooney, T. M., Fox, A. N., Adalbert, R., Powell, E. H., Sheehan, A. E., Avery, M. A., Hackett, R., Logan, M. A., MacDonald, J. M., et al. (2012). dSarm/Sarm1 is required for activation of an injury-induced axon death pathway. Science 337, 481-484. 647
Gerdts, J., Summers, D. W., Sasaki, Y., DiAntonio, A., and Milbrandt, J. (2013). Sarm1-mediated axon degeneration requires both SAM and TIR interactions. J Neurosci 33, 13569-13580.
Fernandes, K. A., Mitchell, K. L., Patel, A., Marola, O. J., Shrager, P., Zack, D. J., Libby, R. T., and Welsbie, D. S. (2018). Role of SARM1 and DR6 in retinal ganglion cell axonal and somal degeneration following axonal injury. Experimental eye research 171, 54-61.
Lautrup, S., Sinclair, D. A., Mattson, M. P., and Fang, E. F. (2019). NAD(+) in Brain Aging and Neurodegenerative Disorders. Cell metabolism 30, 630-655.
Lin, J. B., Kubota, S., Ban, N., Yoshida, M., Santeford, A., Sene, A., Nakamura, R., Zapata, N., Kubota, M., Tsubota, K., Yoshino, J., et al. (2016). NAMPT-Mediated NAD(+) Biosynthesis Is Essential for Vision In Mice. Cell reports 17, 69-85.
Lin, J. B., and Apte, R. S. (2018). NAD(+) and sirtuins in retinal degenerative diseases: A look at future therapies. Prog Retin Eye Res 67, 118-129.
Yang, L., Li, S., Miao, L., Huang, H., Liang, F., Teng, X., Xu, L., Wang, Q., Xiao, W., Ridder, W. H., 3rd, Ferguson, T. A., et al. (2016). Rescue of Glaucomatous Neurodegeneration by Differentially Modulating Neuronal Endoplasmic Reticulum Stress Molecules. J Neurosci 36, 5891-5903.
Yang, L., Miao, L., Liang, F., Huang, H., Teng, X., Li, S., Nuriddinov, J., Selzer, M. E., and Hu, Y. (2014). The mTORC1 effectors S6K1 and 4E-BP play different roles in CNS axon regeneration. Nature communications 5, 5416.
Miao, L., Yang, L., Huang, H., Liang, F., Ling, C., and Hu, Y. (2016). mTORC1 is necessary but mTORC2 and GSK3beta are inhibitory for AKT3-induced axon regeneration in the central nervous system. eLife 5, e14908.
Huang, H., Miao, L., Yang, L., Liang, F., Wang, Q., Zhuang, P., Sun, Y., and Hu, Y. (2019). AKT-dependent and-independent pathways mediate PTEN deletion-induced CNS axon regeneration. Cell death & disease 10, 203.
Huang, H., Miao, L., Liang, F., Liu, X., Xu, L., Teng, X., Wang, Q., Ridder, W. H., 3rd, Shindler, K. S., Sun, Y., and Hu, Y. (2017). Neuroprotection by elF2alpha-CHOP inhibition 677 and XBP-1 activation in EAE/optic neuritis. Cell death & disease 8, e2936.
Chou, T. H., Bohorquez, J., Toft-Nielsen, J., Ozdamar, O., and Porciatti, V. (2014). Robust mouse pattern electroretinograms derived simultaneously from each eye using a common snout electrode. Invest Ophthalmol Vis Sci 55, 2469-2475.
Porciatti, V. (2015). Electrophysiological assessment of retinal ganglion cell function. Experimental eye research 141, 164-170.
Prusky, G. T., Alam, N. M., Beekman, S., and Douglas, R. M. (2004). Rapid quantification of adult and developing mouse spatial vision using a virtual optomotor system. Invest Ophthalmol Vis Sci 45, 4611-4616.
Douglas, R. M., Alam, N. M., Silver, B. D., McGill, T. J., Tschetter, W. W., and Prusky, G. T. (2005). Independent visual threshold measurements in the two eyes of freely moving rats and mice using a virtual-reality optokinetic system. Vis Neurosci 22, 677-684.

Sequences

(SEQ ID NO: 1) mSncg-1.45 kb promoter
ccattgacgtcaataatgacgtatgttcccatagtaacgccaatagggactttccattgacgtcaatgggggagtatttacggtaa
actgcccacttggcagtacatcaagtgtatcatatgggtcccatgccactagtgggagctgtgttacctgttgcagccccacccaa
agcccctgctataggtcaagcaggaatcaccctgccatccccagcctggggcctggagtaccagatccaggaaactagcatcc
cttagctatagagatagccacacatcagcccattcctcagatgtgtatctggggctcagacatcatctcccgatctccgacaaggg
caggatttccttaccgtctgatggggtctctgctggtatcctcagcccctagtctccagccttcaggcatgccgggcctattgagatgg
gagaacttggtaccggggtcctgtgcccaggaccctagcagtccccagctcaggtacaccccaaagcccagcagcagtgtcg
ggatcatggtgaggggctcctgtgctgatgctcagccttacaagtgactctcaaatttgctggtgatgtggtcttcaagcgaaatgtc
agaaagaaaagaaaacacgaggacaacaaagggaggaagtggcctggtccggcccacccggcaagtctcatccgccccc
gcccccgccccttccagcctggcccccttggaggcctccaaccactcaggtcaattcctgtgtcctgagggcacttgaatcaggg
acacgggatttggtagacacataaaggtggccccattaaacttatttctccaggactctgtcgtgggcctggaggagatctggtgc
cacccatactgttggccaggaagtggggaacgggcacatcacacctgctcggcaccttgggctatgggaactagcaggtgggt
gggaactcagagaaggaaagggactatgctagaatcacacagcgggcagcccagtctagggcatggggagcagctttgggt
gtttctggcctcagccttccaacaggtttggctagagctccaggctcaagagcatccaggatacagtggggaactggataacag
ggcagcctgcaggttggccattcattggattggccctgaccccggcccagcctgggacactgaggcatcatcagtcaaggcactt
ttcttctgcatataagagccagggcacgagaccaccagggctttccaaggatgaatgaggtgtaatgatagattaggatatgtcc
agcctccaacacgctctccctcccccagggccaacaagagtcagcagggcagaatagagccagtaggggcccgggccctg
ctcgctggtatccccgtgaggcatgccttctctctggcccgccctccctgcccccaccctggcccgggctggctgggctccagcca
gcagccacagcatcaatatttcatctgcgtcaataagaggcagtagcagcagagacagcggctgcggcagcactccagtccat
agcttgcagcagccaggttccatccttgcaaacaccatggacgtcttcaag
(SEQ ID NO: 2) mSncg-1.03b promoter
ccattgacgtcaataatgacgtatgttcccatagtaacgccaatagggactttccattgacgtcaatgggtggagtatttacggtaa
actgcccacttggcagtacatcaagtgtatcatatgtcagccttacaagtgactctcaaatttgctggtgatgtggtcttcaagcgaa
atgtcagaaagaaaagaaaacacgaggacaacaaagggaggaagtggcctggtccggcccacccggcaagtctcatccg
cccccgcccccgccccttccagcctggcccccttggaggcctccaaccactcaggtcaattcctgtgtcctgagggcacttgaatc
agggacacgggatttggtagacacataaaggtggccccattaaacttatttctccaggactctgtcgtgggcctggaggagatctg
gtgccacccatactgttggccaggaagtggggaacgggcacatcacacctgctcggcaccttgggctatgggaactagcaggt
gggtgggaactcagagaaggaaagggactatgctagaatcacacagcgggcagcccagtctagggcatggggagcagcttt
gggtgtttctggcctcagccttccaacaggtttggctagagctccaggctcaagagcatccaggatacagtggggaactggataa
cagggcagcctgcaggttggccattcattggattggccctgaccccggcccagcctgggacactgaggcatcatcagtcaaggc
acttttcttctgcatataagagccagggcacgagaccaccagggctttccaaggatgaatgaggtgtaatgatagattaggatatg
tccagcctccaacacgctctccctcccccagggccaacaagagtcagcagggcagaatagagccagtaggggcccgggccc
tgctcgctggtatccccgtgaggcatgccttctctctggcccgccctccctgcccccaccctggcccgggctggctgggctccagc
cagcagccacagcatcaatatttcatctgcgtcaataagaggcagtagcagcagagacagcggctgcggcagcactccagtc
catagcttgcagcagccaggttccatccttgcaaacaccatggacgtcttcaag
(SEQ ID NO: 3) mSncg-0.66 kb promoter
ccattgacgtcaataatgacgtatgttoccatagtaacgccaatagggactttccattgacgtcaatgggtggagtatttacggtaa
actgcccacttggcagtacatcaagtgtatcatatgctatgggaactagcaggtggggggaactcagagaaggaaagggact
atgctagaatcacacagcgggcagcccagtctagggcatggggagcagctttgggtgtttctggcctcagccttccaacaggttt
ggctagagctccaggctcaagagcatccaggatacagtggggaactggataacagggcagcctgcaggttggccattcattgg
attggccctgaccccggcccagcctgggacactgaggcatcatcagtcaaggcacttttcttctgcatataagagccagggcacg
agaccaccagggctttccaaggatgaatgaggtgtaatgatagattaggatatgtccagcctccaacacgctctccctcccccag
ggccaacaagagtcagcagggcagaatagagccagtaggggcccgggccctgctcgctggtatccccgtgaggcatgccttc
tctctggcccgccctccctgcccccaccctggcccgggctggctgggctccagccagcagccacagcatcaatatttcatctgcgt
caataagaggcagtagcagcagagacagcggctgcggcagcactccagtccatagcttgcagcagccaggttccatccttgc
aaacaccatggacgtcttcaag
(SEQ ID NO: 4) mSncg-0.27 kb promoter
ccattgacgtcaataatgacgtatgttcccatagtaacgccaatagggactttccattgacgtcaatgggtggagtatttacggtaa
actgcccacttggcagtacatcaagtgtatcatatgaagagtcagcagggcagaatagagccagtaggggcccgggccctgct
cgctggtatccccgtgaggcatgccttctctctggcccgccctccctgcccccaccctggcccgggctggctgggctccagccag
cagccacagcatcaatatttcatctgcgtcaataagaggcagtagcagcagagacagcggctgcggcagcactccagtccata
gcttgcagcagccaggttccatccttgcaaacaccatggacgtcttcaag
(SEQ ID NO: 5) human NMNAT2 full-length
MTETTKTHVI LLACGSFNPI TKGHIQMFER ARDYLHKTGR FIVIGGIVSP VHDSYGKQGL
VSSRHRLIMC QLAVQNSDWI RVDPWECYQD TWQTTCSVLE HHRDLMKRVT
GCILSNVNTP SMTPVIGQPQ NETPQPIYQN SNVATKPTAA KILGKVGESL SRICCVRPPV
ERFTFVDENA NLGTVMRYEE IELRILLLCG SDLLESFCIP GLWNEADMEV IVGDFGIVVV
PRDAADTDRI MNHSSILRKY KNNIMVVKDD INHPMSVVSS TKSRLALQHG DGHVVDYLSQ
PVIDYILKSQ LYINASG
SEQ ID NO: 6 human NMNAT2 A exon 6
MTETTKTHVI LLACGSFNPI TKGHIQMFER ARDYLHKTGR FIVIGGIVSP VHDSYGKQGL
VSSRHRLIMC QLAVQNSDWI RVDPWECYQD TWQTTCSVLE HHRDLMKRVT
GCILSNVNTP SMTPVIGQPQ NETPQPIYQN SNVATKPTAA ENA NLGTVMRYEE
IELRILLLCG SDLLESFCIP GLWNEADMEV IVGDFGIVVV PRDAADTDRI MNHSSILRKY
KNNIMVVKDD INHPMSVVSS TKSRLALQHG DGHVVDYLSQ PVIDYILKSQ LYINASG
SEQ ID NO: 7 Mouse NMNAT2 full length CDS:
ATGACCGAGACCACAAAGACCCACGTTATCCTGCTGGCCTGCGGCAGCTTCAATCCCA
TCACTAAAGGGCACATTCAGATGTTCGAGAGAGCCAGGGATTATCTGCACAAGACTGG
AAGATTTATTGTGATTGGGGGGATTGTCTCTCCGGTCCATGACTCCTACGGAAAACAGG
GCCTTGTGTCAAGTCGGCACCGTCTCATCATGTGTCAGCTGGCTGTCCAGAATTCCGA
CTGGATCAGGGTGGACCCATGGGAGTGCTATCAGGACACCTGGCAGACAACCTGCAG
TGTGTTGGAGCACCATCGAGACCTGATGAAGAGGGTGACCGGCTGCATCCTCTCCAAC
GTCAACACACCTTCCATGACACCTGTGATCGGACAGCCACAGCATGAGAACACCCAGC
CCATTTACCAGAACAGCAATGTGCCCACCAAGCCCACTGCAGCCAAGATCTTGGGAAA
GGTGGGAGAAAGCCTCAGCCGGATCTGCTGCGTCCGCCCACCAGTGGAGCGCTTCAC
TTTTGTAGATGAGAACGCCAACCTGGGCACAGTGATGCGGTATGAGGAGATCGAGCTG
CGCATCTTGCTGCTGTGTGGTAGTGACCTGCTGGAGTCTTTCTGCATCCCAGGACTCT
GGAATGAGGCAGATATGGAAGTGATTGTTGGGGACTTTGGGATCGTCGTGGTACCCAG
GGATGCAGCGGACACAGACCGGATCATGAATCACTCCTCCATACTCCGCAAGTACAAA
AACAACATCATGGTTGTGAAGGATGATATCAACCATCCCATGTCTGTAGTCAGCTCCAC
CAAGAGCAGGCTGGCCCTGCAGCATGGGGACGGCCATGTTGTGGATTACCTGTCCCA
GCCAGTCATCGATTACATCCTCAAGAGTCAGCTGTACATCAACGCCTCGGGCTAG
SEQ ID NO: 8 Mouse NMNAT2 full length protein seq:
MTETTKTHVILLACGSFNPITKGHIQMFERARDYLHKTGRFIVIGGIVSPVHDSYGKQGLVSS
RHRLIMCQLAVQNSDWIRVDPWECYQDTWQTTCSVLEHHRDLMKRVTGCILSNVNTPSMT
PVIGQPQHENTQPIYQNSNVPTKPTAAKILGKVGESLSRICCVRPPVERFTFVDENANLGTV
MRYEEIELRILLLCGSDLLESFCIPGLWNEADMEVIVGDFGIVVVPRDAADTDRIMNHSSILRK
YKNNIMVVKDDINHPMSVVSSTKSRLALQHGDGHVVDYLSQPVIDYILKSQLYINASG
SEQ ID NO: 9 Mouse NMNAT2 A exon 6 CDS:
Atgaccgagaccacaaagacccacgttatcctgctggcctgcggcagcttcaatcccatcactaaagggcacattcagatgttc
gagagagccagggattatctgcacaagactggaagatttattgtgattggcgggattgtctctccggtccatgactcctacggaaa
acagggccttgtgtcaagtcggcaccgtctcatcatgtgtcagctggctgtccagaattccgactggatcagggggacccatggg
agtgctatcaggacacctggcagacaacctgcagtgtgttggagcaccatcgagacctgatgaagagggtgaccggctgcatc
ctctccaacgtcaacacaccttccatgacacctgtgatoggacagccacagcatgagaacacccagcccatttaccagaacag
caatgtgcccaccaagcccactgccgcggagaacgccaacctgggcacagtgatgcggtatgaggagatcgagctgcgcat
cttgctgctgtgtggtagtgacctgctggagtctttctgcatcccaggactctggaatgaggcagatatggaagtgattgttggggac
tttgggatcgtcgtggtacccagggatgcagcggacacagaccggatcatgaatcactcctccatactccgcaagtacaaaaac
aacatcatggttgtgaaggatgatatcaaccatcccatgtctgtagtcagctccaccaagagcaggctggccctgcagcatggg
gacggccatgttgtggattacctgtcccagccagtcatcgattacatcctcaagagtcagctgtacatcaacgcctcgggctag
SEQ ID NO: 10 Mouse NMNAT2 A exon 6 protein seq:
MTETTKTHVILLACGSFNPITKGHIQMFERARDYLHKTGRFIVIGGIVSPVHDSYGKQGLVSS
RHRLIMCQLAVQNSDWIRVDPWECYQDTWQTTCSVLEHHRDLMKRVTGCILSNVNTPSMT
PVIGQPQHENTQPIYQNSNVPTKPTAAENANLGTVMRYEEIELRILLLCGSDLLESFCIPGLW
NEADMEVIVGDFGIVVVPRDAADTDRIMNHSSILRKYKNNIMVVKDDINHPMSVVSSTKSRL
ALQHGDGHVVDYLSQPVIDYILKSQLYINASG
SEQ ID NO: 11 AAV Vector with mouse NMNAT2 sequence in bold
ctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgctactt
atttacgtagccatgctctaggtaccattgacgtcaataatgacgtatgttcccatagtaacgccaatagggactttccattgacgtc
aatgggtggagtatttacggtaaactgcccacttggcagtacatcaagtgtatcatatggtcccatgccactagtgggagctgtgtt
acctgttgcagccccacccaaagcccctgctataggtcaagcaggaatcaccctgccatccccagcctggggcctggagtacc
agatccaggaaactagcatcccttagctatagagatagccacacatcagcccattcctcagatgtgtatctggggctcagacatc
atctcccgatctccgacaagggcaggatttccttaccgtctgatggggtctctgctggtatcctcagcccctagtctccagccttcag
gcatgccgggcctattgagatgggagaacttggtaccggggtcctgtgcccaggaccctagcagtccccagctcaggtacaccc
caaagcccagcagcagtgtcgggatcatggtgaggggctcctgtgctgatgctcagccttacaagtgactctcaaatttgctggtg
atgtggtcttcaagcgaaatgtcagaaagaaaagaaaacacgaggacaacaaagggaggaagtggcctggtccggcccac
ccggcaagtctcatccgcccccgcccccgccccttccagcctggcccccttggaggcctccaaccactcaggtcaattcctgtgtc
ctgagggcacttgaatcagggacacgggatttggtagacacataaaggtggccccattaaacttatttctccaggactctgtcgtg
ggcctggaggagatctggtgccacccatactgttggccaggaagtggggaacgggcacatcacacctgctcggcaccttgggc
tatgggaactagcaggtgggtgggaactcagagaaggaaagggactatgctagaatcacacagcgggcagcccagtctagg
gcatggggagcagctttgggtgtttctggcctcagccttccaacaggtttggctagagctccaggctcaagagcatccaggatac
agtggggaactggataacagggcagcctgcaggttggccattcattggattggccctgaccccggcccagcctgggacactga
ggcatcatcagtcaaggcacttttcttctgcatataagagccagggcacgagaccaccagggctttccaaggatgaatgaggtgt
aatgatagattaggatatgtccagcctccaacacgctctccctcccccagggccaacaagagtcagcagggcagaatagagc
cagtaggggcccgggccctgctcgctggtatccccgtgaggcatgccttctctctggcccgccctccctgcccccaccctggccc
gggctggctgggctccagccagcagccacagcatcaatatttcatctgcgtcaataagaggcagtagcagcagagacagcgg
ctgcggcagcactccagtccatagcttgcagcagccaggttccatccttgcaaacaccatggacgtcttcaaggaattgaattgg
atccgccaccatgtacccatacgatgttcctgactatgcgggctatccgtatgacgtcccggactatgcagggtcctatccatatga
cgttccagattacgctagatctgaattgatgaccgagaccacaaagacccacgttatcctgctggcctgcggcagcttcaa
tcccatcactaaagggcacattcagatgttcgagagagccagggattatctgcacaagactggaagatttattgtgattg
gcgggattgtctctccggtccatgactcctacggaaaacagggccttgtgtcaagtcggcaccgtctcatcatgtgtca
gctggctgtccagaattccgactggatcagggtggacccatgggagtgctatcaggacacctggcagacaacctgca
gtgtgttggagcaccatcgagacctgatgaagagggtgaccggctgcatcctctccaacgtcaacacaccttccatga
cacctgtgatcggacagccacagcatgagaacacccagcccatttaccagaacagcaatgtgcccaccaagcccac
tgccgcggagaacgccaacctgggcacagtgatgcggtatgaggagatcgagctgcgcatcttgctgctgtgtggta
gtgacctgctggagtctttctgcatcccaggactctggaatgaggcagatatggaagtgattgttggggactttgggatc
gtcgtggtacccagggatgcagcggacacagaccggatcatgaatcactcctccatactccgcaagtacaaaaaca
acatcatggttgtgaaggatgatatcaaccatcccatgtctgtagtcagctccaccaagagcaggctggccctgcagc
atggggacggccatgttgtggattacctgtcccagccagtcatcgattacatcctcaagagtcagctgtacatcaacgc
ctcgggctagacgcgtctcgagaagcttatcgataatcaacctctggattacaaaatttgtgaaagattgactggtattottaactat
gttgctccttttacgctatgtggatacgctgctttaatgcctttgtatcatgctattgottcccgtatggctttcattttctcctccttgtat
aaatcctggttgctgtctctttatgaggagttgtggcccgttgtcaggcaacgtggcgtggtgtgcactgtgtttgctgacgcaacccccact
ggttggggcattgccaccacctgtcagctcctttccgggactttcgctttccccctccctattgccacggcggaactcatcgccgcct
gccttgcccgctgctggacaggggctcggctgttgggcactgacaattccgtggtgttgtcggggaaatcatcgtcctttccttggct
gctcgcctgtgttgccacctggattctgcgcgggacgtccttctgctacgtcccttcggccctcaatccagcggaccttccttcccgc
ggcctgctgccggctctgcggcctcttccgcgtcttcgccttcgccctcagacgagtcggatctccctttgggccgcctccccgcatc
gataccgtcgactcgctgatcagcctcgactgtgccttctagttgccagccatctgttgtttgcccctcccccgtgccttccttgaccct
ggaaggtgccactcccactgtcctttcctaataaaatgaggaaattgcatcgcattgtctgagtaggtgtcattctattctggggggtg
gggtggggcaggacagcaagggggaggattgggaagacaatagcaggcatgctggggatgcggtgggctctatggcttctg
aggcggaaagaaccagctggggctcgactagagcatggctacgtagataagtagcatggcgggttaatcattaactacaagg
aacccctagtgatggagttggccactccctctctgcgcgctcgctcgctcactgaggccgggcgaccaaaggtcgcccgacgcc
cgggctttgcccgggggcctcagtgagcgagcgagcgcgcagCTAGAATTAATTCCGTGTATTCTATAGT
GTCACctaAATCGTATGTGTATGATACATAAGGTTATGTATTAATTGTAGCCGCGTTCTAAC
GACAATATGTACAAGCCTAATTGTGTAGCATCTGGCTTACTGAAGCAGACCCTATCATCT
CTCTCGTAAACTGCCGTCAGAGTCGGTTTGGTTGGACGAACCTTCTGAGTTTCTGGTAA
CGCCGTCCCGCACCCGGAAATGGTCAGCGAACCAATCAGCAGGGTCATCGCTAGCCA
GATCCTCTACGCCGGACGCATCGTGGCCGGCATCACCGGCGCCACAGGTGCGGTTGC
TGGCGCCTATATCGCCGACATCACCGATGGGGAAGATCGGGCTCGCCACTTCGGGCT
CATGAGCGCTTGTTTCGGCGTGGGTATGGTGGCAGGCCCCGTGGCCGGGGGACTGTT
GGGCGCCATCTCCTTGCATGCACCATTCCTTGCGGCGGCGGTGCTCAACGGCCTCAAC
CTACTACTGGGCTGCTTCCTAATGCAGGAGTCGCATAAGGGAGAGCGTCGAATGGTGC
ACTCTCAGTACAATCTGCTCTGATGCCGCATAGTTAAGCCAGCCCCGACACCCGCCAA
CACCCGCTGACGCGCCCTGACGGGCTTGTCTGCTCCCGGCATCCGCTTACAGACAAG
CTGTGACCGTCTCCGGGAGCTGCATGTGTCAGAGGTTTTCACCGTCATCACCGAAACG
CGCGAGACGAAAGGGCCTCGTGATACGCCTATTTTTATAGGTTAATGTCATGATAATAA
TGGTTTCTTAGACGTCaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaacatttccgtgtc
gcccttattcccttttttgcggcattttgccttcctgtttttgctcacccagaaacgctggtgaaagtaaaagatgctgaagatcagttgg
gtgcacgagtgggttacatcgaactggatctcaacagcggtaagatccttgagagttttcgccccgaagaacgttttccaatgatg
agcacttttaaagttctgctatgtggcgcggtattatcccgtattgacgccgggcaagagcaactcggtcgccgcatacactattctc
agaatgacttggttgagtactcaccagtcacagaaaagcatcttacggatggcatgacagtaagagaattatgcagtgctgccat
aaccatgagtgataacactgcggccaacttacttctgacaacgatcggaggaccgaaggagctaaccgcttttttgcacaacat
gggggatcatgtaactcgccttgatcgttgggaaccggagctgaatgaagccataccaaacgacgagcgtgacaccacgatg
cctgtagcaatggcaacaacgttgcgcaaactattaactggcgaactacttactctagcttcccggcaacaattaatagactggat
ggaggcggataaagttgcaggaccacttctgcgctcggcccttccggctggctggtttattgctgataaatctggagccggtgagc
gtgggtctcgcggtatcattgcagcactggggccagatggtaagccctcccgtatcgtagttatctacacgacggggagtcaggc
aactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaac
gtgagttttcgttccactgagcgtcagaccccgtagaaaagatcaaaggatottcttgagatcctttttttctgcgcgtaatctgctgctt
gcaaacaaaaaaaccaccgctaccagcggtggtttgtttgccggatcaagagctaccaactctttttccgaaggtaactggcttca
gcagagcgcagataccaaatactgttcttctagtgtagccgtagttaggccaccacttcaagaactctgtagcaccgcctacatac
ctcgctctgctaatcctgttaccagtggctgctgccagtggcgataagtcgtgtcttaccgggttggactcaagacgatagttaccgg
ataaggcgcagcggtcgggctgaacggggggttcgtgcacacagcccagcttggagcgaacgacctacaccgaactgagat
acctacagcgtgagctatgagaaagcgccacgcttcccgaagggagaaaggcggacaggtatccggtaagcggcagggtc
ggaacaggagagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttcctggcctttt
gctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcccaatacgcaaaccgcctctccccg
cgcgttggccgattcattaatgcagctgTGGAATGTGTGTCAGTTAGGGTGTGGAAAGTCCCCAGGCT
CCCCAGCAGGCAGAAGTATGCAAAGCATGCATCTCAATTAGTCAGCAACCAGGTGTGG
AAAGTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCGCC
TCGGCCTCTGAGCTATTCCAGAAGTAGTGAGGAGGCTTTTTTGGAGGCCTAGGCTTTTG
CAAAAAGCT

SEQ ID NO: 12, 13, 14 and 15 are provided in the sequence listing, and correspond to codon optimized DNA sequence encoding human NMNAT2 (SEQ ID NO:12), codon optimized DNA sequence encoding human NMNAT2 Δ exon 6 (SEQ ID NO:13); a therapeutic AAV vector comprising codon optimized DNA sequence encoding human NMINAT2 (SEQ ID NO:14) and an AAV vector comprising codon optimized DNA sequence encoding human NMNAT2 Δ exon 6 (SEQ ID NO:15).

(SEQ ID NO: 12): The coding sequence (CDS) of
codon optimized human NMNAT2 gene full length
(cophNMNAT2)
ATGACAGAGACCACCAAGACCCATGTGATCCTGCTGGCCTGTGGCAGCT
TCAACCCCATCACCAAGGGCCACATCCAGATGTTTGAAAGAGCCAGAGA
CTACCTGCACAAGACAGGCAGATTCATTGTGATTGGAGGCATAGTGAGC
CCTGTGCATGACTCCTATGGCAAGCAAGGCCTGGTGAGCAGCAGACACA
GACTGATCATGTGCCAACTGGCTGTCCAGAACTCTGACTGGATCAGGGT
GGACCCTTGGGAATGCTACCAGGACACCTGGCAGACCACCTGCTCTGTC
CTGGAACACCACAGAGACCTGATGAAGAGGGTGACAGGCTGCATCCTGA
GCAATGTGAACACCCCTAGCATGACCCCTGTGATTGGACAGCCTCAGAA
TGAGACCCCTCAGCCCATCTACCAGAACAGCAATGTGGCCACCAAGCCT
ACAGCTGCCAAGATCCTGGGCAAAGTGGGAGAGAGCCTTAGCAGGATCT
GCTGTGTGAGGCCCCCAGTTGAGAGATTCACATTTGTGGATGAGAATGC
CAACCTGGGCACAGTGATGAGATATGAGGAGATTGAACTGAGAATCCTG
CTCCTGTGTGGCTCTGACCTGCTGGAGTCCTTCTGCATCCCTGGCCTGT
GGAATGAGGCTGACATGGAAGTGATTGTGGGAGACTTTGGCATAGTGGT
GGTCCCTAGAGATGCTGCAGACACAGATAGAATCATGAACCACAGCTCC
ATCCTGAGAAAATACAAGAACAACATCATGGTGGTGAAGGATGACATCA
ACCACCCCATGTCTGTGGTGAGCAGCACCAAGAGCAGACTGGCCCTGCA
GCATGGTGATGGCCATGTGGTGGACTACCTGAGCCAGCCAGTGATAGAT
TACATCCTGAAAAGCCAGCTGTACATCAATGCCTCTGGCTGA
(SEQ ID NO: 13): The coding sequence (CDS) of
codon optimized human NMNAT2 Δexon6
(cophNMNAT2ΔEx6)
ATGACAGAGACCACCAAGACCCATGTGATCCTGCTGGCCTGTGGCAGCT
TCAACCCCATCACCAAGGGCCACATCCAGATGTTTGAAAGAGCCAGAGA
CTACCTGCACAAGACAGGCAGATTCATTGTGATTGGAGGCATAGTGAGC
CCTGTGCATGACTCCTATGGCAAGCAAGGCCTGGTGAGCAGCAGACACA
GACTGATCATGTGCCAACTGGCTGTCCAGAACTCTGACTGGATCAGGGT
GGACCCTTGGGAATGCTACCAGGACACCTGGCAGACCACCTGCTCTGTC
CTGGAACACCACAGAGACCTGATGAAGAGGGTGACAGGCTGCATCCTGA
GCAATGTGAACACCCCTAGCATGACCCCTGTGATTGGACAGCCTCAGAA
TGAGACCCCTCAGCCCATCTACCAGAACAGCAATGTGGCCACCAAGCCT
ACAGCTGCCGAGAATGCCAACCTGGGCACAGTGATGAGATATGAGGAGA
TTGAACTGAGAATCCTGCTCCTGTGTGGCTCTGACCTGCTGGAGTCCTT
CTGCATCCCTGGCCTGTGGAATGAGGCTGACATGGAAGTGATTGTGGGA
GACTTTGGCATAGTGGTGGTCCCTAGAGATGCTGCAGACACAGATAGAA
TCATGAACCACAGCTCCATCCTGAGAAAATACAAGAACAACATCATGGT
GGTGAAGGATGACATCAACCACCCCATGTCTGTGGTGAGCAGCACCAAG
AGCAGACTGGCCCTGCAGCATGGTGATGGCCATGTGGTGGACTACCTGA
GCCAGCCAGTGATAGATTACATCCTGAAAAGCCAGCTGTACATCAATGC
CTCTGGCTGA

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A composition comprising:
a mammalian AAV vector, which comprises:
a γ-synuclein (Sncg) promoter, or functional fragment thereof, that promotes expression of an operably linked coding sequence specifically in retinal ganglion cells (RGCs), and
a sequence encoding a functional human nicotinamide mononucleotide adenylyl transferase 2 (NMNTA2) protein, or a variant thereof.

2. The composition of claim 1 wherein the NMNTA2 protein has an extended half-life in vivo compared to the wild-type protein.

3. The composition of claim 1, wherein the NMNTA2 protein comprises an exon 6 deletion.

4. The composition of claim 1, wherein the NMNTA2 protein is a wild-type protein.

5. The composition of claim 1, wherein the NMNTA2 protein has at least 95% sequence identity to SEQ ID NO:5 or SEQ ID NO:6.

6. The composition of claim 1, wherein the promoter is a murine Sncg promoter.

7. The composition of claim 6, wherein the promoter comprises a sequence of any of SEQ ID NO:1-4, or a variant thereof.

8. The composition of claim 7, wherein a promoter variant has at least 95% sequence identity to SEQ ID NO:1, 2, 3, or 4.

9. The composition of claim 1, wherein the mammalian vector is a mammalian viral vector.

10. An AAV virus particle comprising a vector of claim 1.

11. A method of treating an optic nerve (ON) neuropathy in a mammalian subject in need thereof, the method comprising:
intravitreally administering the composition of any claim 1 into the subject, thereby treating the ON neuropathy.

12. A method of reducing or ameliorating degeneration of axons and/or soma of RGCs, comprising:
intravitreally administering the composition of claim 1 into a mammalian subject experiencing or at imminent risk of an ON neuropathy.

13. The method of claim 11, wherein the ON neuropathy is retinal ganglion cell degeneration, including glaucoma, optic neuritis, ON traumatic injury and other ON-related diseases.

14. The method of claim 13, wherein the ON neuropathy is glaucoma.

15. The method of claim 14, wherein the subject is human.

16. The composition of claim 1, wherein the vector has a sequence of any of SEQ ID NO:11, SEQ ID NO:14 and SEQ ID NO:15
